SEQUENCE LISTING (12 sequences)

SEQ ID NO: 1 - DNA, 4000 bp, Artificial Sequence
  West Nile Delta C - rabies PIV sequence (partial)
agtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtcaatatgctaaaacgcggaatgccccgcgtgttgtccttgattggacttaagcaaaagaagcgagggggcaagactggtatagctgtgatcgttcctcaggctcttttgtttgtacccttgctggtatttcccctttgctttggtaaatttcctatctataccatccctgataagctcgggccttggagtcccattgatattcaccatttgagctgcccaaacaacctcgtcgttgaggatgaagggtgcactaatctttctggattttcctacatggagttgaaagtgggctatatttcagccattaagatgaacggctttacttgtacaggagtcgtgaccgaagccgagacatatacaaatttcgtgggatacgtcaccaccaccttcaagagaaaacacttccgcccaacgcctgacgcttgtcgggccgcttacaactggaagatggcaggagatcctcgatatgaagaatctctgcacaacccgtatcctgattaccattggctgcggacagtcaagactaccaaggagagtctggtcattatatcaccaagcgtggccgatcttgatccttatgatagatccctgcacagtagggtttttcctggcgggaattgtagcggtgttgcagtatcaagtacctactgctccactaaccacgactacactatatggatgcctgagaaccctcgactcggtatgagttgcgacatttttacgaactcacggggcaagcgggcatctaaggggtctgaaacatgcgggtttgttgatgagcgggggttgtataaatctcttaaaggcgcctgtaagctgaaactctgtggcgtactggggctgcgcctgatggacggcacatgggtggctatgcagacaagcaatgaaacaaagtggtgtccccctggtcagctggttaatctgcacgactttaggtctgacgaaatcgagcaccttgtggtggaggaactggtgaagaaacgcgaagagtgcctggacgcacttgagagtattatgaccaccaaatccgtttccttcagaagactgagccacctgcgaaagctggtgccagggttcgggaaggcttatactattttcaacaagactcttatggaggcggatgcccattataagtcagttaggacttggaatgagataattccctccaaaggatgtctgagagtcggtgggagatgccacccccatgtcaatggggtgttctttaacggaatcatcctgggacctgacgggaacgtgctgattcccgagatgcaatcttcccttctgcagcaacacatggaactcctggtgtcttcagtgatacccctgatgcacccactggccgaccccagcactgtgttcaaaaatggcgatgaggccgaagactttgtggaagttcacctgcccgatgtacacgaaaggatatctggagtagacctgggccttcctaattggggtaagtacgtgctcctgagtgcgggtgccttgaccgctttgatgctgatcatttttctgatgacctgctggcggagggtgaatcgctccgagccgacacagcacaatctcagagggacaggccgggaagtaagtgtgactccgcaatctggcaagattattagtagttgggagagttacaagtctggaggagagactgggttgaattttgatctgctcaaacttgcaggcgatgtagaatcaaatcctggacccggaggaaagaccggtattgcagtcatgattggcctgatcgcctgcgtaggagcagttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacttatgaatgcccagtgctgtcggctggtaatgatccagaagacatcgactgttggtgcacaaagtcagcagtctacgtcaggtatggaagatgcaccaagacacgccactcaagacgcagtcggaggtcactgacagtgcagacacacggagaaagcactctagcgaacaagaagggggcttggatggacagcaccaaggccacaaggtatttggtaaaaacagaatcatggatcttgaggaaccctggatatgccctggtggcagccgtcattggttggatgcttgggagcaacaccatgcagagagttgtgtttgtcgtgctattgcttttggtggccccagcttacagctttaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggccaacctggcagaggtccgcagttattgctatttggctaccgtcagcgatctctccaccaaagctgcgtgcccggccatgggagaagctcacaatgacaaacgtgctgacccagcttttgtgtgcagacaaggagtggtggacaggggctggggcaacggctgcggactatttggcaaaggaagcattgacacatgcgccaaatttgcctgctctaccaaggcaataggaagaaccattttgaaagagaatatcaagtacgaagtggccatttttgtccatggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagtggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccactggcacaagtctggaagcagcattggcaaagcctttacaaccaccctcaaaggagcgcagagactagccgctctaggagacacagcttgggactttggatcagttggaggggtgttcacctcagttgggaaggctgtccatcaagtgttcggaggagcattccgctcactgttcggaggcatgtcctggataacgcaaggattgctgggggctctcctgttgtggatgggcatcaatgctcgtgacaggtccatagctctcacgtttctcgcagttggaggagttctgctcttcctctccgtgaacgtgcacgctgacactgggtgtgccatagacatcagccggcaagagctgagatgtggaagtggagtgttcatacacaatgatgtggaggcttggatggaccggt

SEQ ID NO: 2 - DNA, 4000 bp, Artificial Sequence
  West Nile Delta C - rabies PIV sequence (partial) (complement)
tcatcaagcggacacactcgactgtttgaatcatcacaaacactcctaattgttgttaattgtgtcacgctcgacaaagaatcgtgcttctagagctacagattctttggtcctcccgggccgttctcggcccgacagttatacgattttgcgccttacggggcgcacaacaggaactaacctgaattcgttttcttcgctcccccgttctgaccatatcgacactagcaaggagtccgagaaaacaaacatgggaacgaccataaaggggaaacgaaaccatttaaaggatagatatggtagggactattcgagcccggaacctcagggtaactataagtggtaaactcgacgggtttgttggagcagcaactcctacttcccacgtgattagaaagacctaaaaggatgtacctcaactttcacccgatataaagtcggtaattctacttgccgaaatgaacatgtcctcagcactggcttcggctctgtatatgtttaaagcaccctatgcagtggtggtggaagttctcttttgtgaaggcgggttgcggactgcgaacagcccggcgaatgttgaccttctaccgtcctctaggagctatacttcttagagacgtgttgggcataggactaatggtaaccgacgcctgtcagttctgatggttcctctcagaccagtaatatagtggttcgcaccggctagaactaggaatactatctagggacgtgtcatcccaaaaaggaccgcccttaacatcgccacaacgtcatagttcatggatgacgaggtgattggtgctgatgtgatatacctacggactcttgggagctgagccatactcaacgctgtaaaaatgcttgagtgccccgttcgcccgtagattccccagactttgtacgcccaaacaactactcgcccccaacatatttagagaatttccgcggacattcgactttgagacaccgcatgaccccgacgcggactacctgccgtgtacccaccgatacgtctgttcgttactttgtttcaccacagggggaccagtcgaccaattagacgtgctgaaatccagactgctttagctcgtggaacaccacctccttgaccacttctttgcgcttctcacggacctgcgtgaactctcataatactggtggtttaggcaaaggaagtcttctgactcggtggacgctttcgaccacggtcccaagcccttccgaatatgataaaagttgttctgagaatacctccgcctacgggtaatattcagtcaatcctgaaccttactctattaagggaggtttcctacagactctcagccaccctctacggtgggggtacagttaccccacaagaaattgccttagtaggaccctggactgcccttgcacgactaagggctctacgttagaagggaagacgtcgttgtgtaccttgaggaccacagaagtcactatggggactacgtgggtgaccggctggggtcgtgacacaagtttttaccgctactccggcttctgaaacaccttcaagtggacgggctacatgtgctttcctatagacctcatctggacccggaaggattaaccccattcatgcacgaggactcacgcccacggaactggcgaaactacgactagtaaaaagactactggacgaccgcctcccacttagcgaggctcggctgtgtcgtgttagagtctccctgtccggcccttcattcacactgaggcgttagaccgttctaataatcatcaaccctctcaatgttcagacctcctctctgacccaacttaaaactagacgagtttgaacgtccgctacatcttagtttaggacctgggcctcctttctggccataacgtcagtactaaccggactagcggacgcatcctcgtcaatgggagagattgaaggttcccttccactactactgccatttacgatgactgcagtgtctacagtagtgctaaggttgtcgacgacctttcttggatacgtaacagtctcgttacctacaccctatgtacacgctactatgatagtgaatacttacgggtcacgacagccgaccattactaggtcttctgtagctgacaaccacgtgtttcagtcgtcagatgcagtccataccttctacgtggttctgtgcggtgagttctgcgtcagcctccagtgactgtcacgtctgtgtgcctctttcgtgagatcgcttgttcttcccccgaacctacctgtcgtggttccggtgttccataaaccatttttgtcttagtacctagaactccttgggacctatacgggaccaccgtcggcagtaaccaacctacgaaccctcgttgtggtacgtctctcaacacaaacagcacgataacgaaaaccaccggggtcgaatgtcgaaattgacggaaccttactcgttgtctctgaagaaccttcctcacagacctcgttgtacccacctaaaccaagagcttccgctgtcgacgcactgatagtacagattcctgttcggatggtagctacacttctactacttatacctccgccggttggaccgtctccaggcgtcaataacgataaaccgatggcagtcgctagagaggtggtttcgacgcacgggccggtaccctcttcgagtgttactgtttgcacgactgggtcgaaaacacacgtctgttcctcaccacctgtccccgaccccgttgccgacgcctgataaaccgtttccttcgtaactgtgtacgcggtttaaacggacgagatggttccgttatccttcttggtaaaactttctcttatagttcatgcttcaccggtaaaaacaggtacctggttgatgacacctcagcgtgcctttgatgaggtgtgtccaacctcggtgagtccgtccctctaagtcgtagtgaggacgccgcggaagtatgtgtgatttcgaacctcttatacctctccactgtcacctgacacttggtgccagtccctaactgtggttacgtatgatgcactactgacaaccttgtttctgcaagaaccaggtagcactcaccaagtacctggagttggagggaacctcgtcacgaccttcatgacacacctccttgtctctctgcaattacctcaaactccttggtgtgcggtgcttcgtcagacactatcgtaacccgagtgttctccctcgagacgtagttcgaaaccgacctcggtaaggacaccttaaaagttcgttgtgacagttcaactgcagcccagtaaacttcacatctcacttctacctttttaacgtcaacttcccttgttggataccgcagacaagtttccgaaagttcaaagaaccctgagggcgtctgtgtccagtgccgtgacaccacaaccttaacgtcatgtgaccgtgcctacctggaacgtttcaaggatagagcagtcaccgaagtaacttgctggattgcggtcacccgtctaaccagtgacagttgggaaaacaaagtcaccggtgccggttgcgattccaggactaacttaaccttggtgggaaacctctgagtatgtatcaccacccgtctcctcttgttgtctagttagtggtgaccgtgttcagaccttcgtcgtaaccgtttcggaaatgttggtgggagtttcctcgcgtctctgatcggcgagatcctctgtgtcgaaccctgaaacctagtcaacctccccacaagtggagtcaacccttccgacaggtagttcacaagcctcctcgtaaggcgagtgacaagcctccgtacaggacctattgcgttcctaacgacccccgagaggacaacacctacccgtagttacgagcactgtccaggtatcgagagtgcaaagagcgtcaacctcctcaagacgagaaggagaggcacttgcacgtgcgactgtgacccacacggtatctgtagtcggccgttctcgactctacaccttcacctcacaagtatgtgttactacacctccgaacctacctggcca

SEQ ID NO: 3 - DNA, 2200 bp, Artificial Sequence
  West Nile Delta prME - Rabies G PIV sequence (partial)
agtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtctatttgctaaaacgcggaatgccccgcgtgttgtccttgattggacttaagagggctatgttgagcctgatcgacggcaaggggccaatacgatttgtgttggctctcttggcgttcttcaggttcacagcaattgctccgacccgagcagtgctggatcgatggagaggtgtgaacaaacaaacagcgatgaaacaccttctgagtttcaagaaggaactagggaccttgaccagtgctatcaatcggcggagctcaaagcaaaagaagcgagggggcaagactggtatagctgtgatcgttcctcaggctcttttgtttgtacccttgctggtatttcccctttgctttggtaaatttcctatctataccatccctgataagctcgggccttggagtcccattgatattcaccatttgagctgcccaaacaacctcgtcgttgaggatgaagggtgcactaatctttctggattttcctacatggagttgaaagtgggctatatttcagccattaagatgaacggctttacttgtacaggagtcgtgaccgaagccgagacatatacaaatttcgtgggatacgtcaccaccaccttcaagagaaaacacttccgcccaacgcctgacgcttgtcgggccgcttacaactggaagatggcaggagatcctcgatatgaagaatctctgcacaacccgtatcctgattaccattggctgcggacagtcaagactaccaaggagagtctggtcattatatcaccaagcgtggccgatcttgatccttatgatagatccctgcacagtagggtttttcctggcgggaattgtagcggtgttgcagtatcaagtacctactgctccactaaccacgactacactatatggatgcctgagaaccctcgactcggtatgagttgcgacatttttacgaactcacggggcaagcgggcatctaaggggtctgaaacatgcgggtttgttgatgagcgggggttgtataaatctcttaaaggcgcctgtaagctgaaactctgtggcgtactggggctgcgcctgatggacggcacatgggtggctatgcagacaagcaatgaaacaaagtggtgtccccctggtcagctggttaatctgcacgactttaggtctgacgaaatcgagcaccttgtggtggaggaactggtgaagaaacgcgaagagtgcctggacgcacttgagagtattatgaccaccaaatccgtttccttcagaagactgagccacctgcgaaagctggtgccagggttcgggaaggcttatactattttcaacaagactcttatggaggcggatgcccattataagtcagttaggacttggaatgagataattccctccaaaggatgtctgagagtcggtgggagatgccacccccatgtcaatggggtgttctttaacggaatcatcctgggacctgacgggaacgtgctgattcccgagatgcaatcttcccttctgcagcaacacatggaactcctggtgtcttcagtgatacccctgatgcacccactggccgaccccagcactgtgttcaaaaatggcgatgaggccgaagactttgtggaagttcacctgcccgatgtacacgaaaggatatctggagtagacctgggccttcctaattggggtaagtacgtgctcctgagtgcgggtgccttgaccgctttgatgctgatcatttttctgatgacctgctggcggagggtgaatcgctccgagccgacacagcacaatctcagagggacaggccgggaagtaagtgtgactccgcaatctggcaagattattagtagttgggagagttacaagtctggaggagagactgggttgaattttgatctgctcaaacttgcaggcgatgtagaatcaaatcctggacccgcccgggacaggtccatagctctcacgtttctcgcagttggaggagttctgctcttcctctccgtgaacgtgcacgctgacactgggtgtgccatagacatcagccggcaagagctgagatgtggaagtggagtgttcatac

SEQ ID NO: 4 - DNA, 2200 bp, Artificial Sequence
  West Nile Delta prME - Rabies G PIV sequence (partial) (complement)
tcatcaagcggacacactcgactgtttgaatcatcacaaacactcctaattgttgttaattgtgtcacgctcgacaaagaatcgtgcttctagagctacagattctttggtcctcccgggccgttctcggcccgacagataaacgattttgcgccttacggggcgcacaacaggaactaacctgaattctcccgatacaactcggactagctgccgttccccggttatgctaaacacaaccgagagaaccgcaagaagtccaagtgtcgttaacgaggctgggctcgtcacgacctagctacctctccacacttgtttgtttgtcgctactttgtggaagactcaaagttcttccttgatccctggaactggtcacgatagttagccgcctcgagtttcgttttcttcgctcccccgttctgaccatatcgacactagcaaggagtccgagaaaacaaacatgggaacgaccataaaggggaaacgaaaccatttaaaggatagatatggtagggactattcgagcccggaacctcagggtaactataagtggtaaactcgacgggtttgttggagcagcaactcctacttcccacgtgattagaaagacctaaaaggatgtacctcaactttcacccgatataaagtcggtaattctacttgccgaaatgaacatgtcctcagcactggcttcggctctgtatatgtttaaagcaccctatgcagtggtggtggaagttctcttttgtgaaggcgggttgcggactgcgaacagcccggcgaatgttgaccttctaccgtcctctaggagctatacttcttagagacgtgttgggcataggactaatggtaaccgacgcctgtcagttctgatggttcctctcagaccagtaatatagtggttcgcaccggctagaactaggaatactatctagggacgtgtcatcccaaaaaggaccgcccttaacatcgccacaacgtcatagttcatggatgacgaggtgattggtgctgatgtgatatacctacggactcttgggagctgagccatactcaacgctgtaaaaatgcttgagtgccccgttcgcccgtagattccccagactttgtacgcccaaacaactactcgcccccaacatatttagagaatttccgcggacattcgactttgagacaccgcatgaccccgacgcggactacctgccgtgtacccaccgatacgtctgttcgttactttgtttcaccacagggggaccagtcgaccaattagacgtgctgaaatccagactgctttagctcgtggaacaccacctccttgaccacttctttgcgcttctcacggacctgcgtgaactctcataatactggtggtttaggcaaaggaagtcttctgactcggtggacgctttcgaccacggtcccaagcccttccgaatatgataaaagttgttctgagaatacctccgcctacgggtaatattcagtcaatcctgaaccttactctattaagggaggtttcctacagactctcagccaccctctacggtgggggtacagttaccccacaagaaattgccttagtaggaccctggactgcccttgcacgactaagggctctacgttagaagggaagacgtcgttgtgtaccttgaggaccacagaagtcactatggggactacgtgggtgaccggctggggtcgtgacacaagtttttaccgctactccggcttctgaaacaccttcaagtggacgggctacatgtgctttcctatagacctcatctggacccggaaggattaaccccattcatgcacgaggactcacgcccacggaactggcgaaactacgactagtaaaaagactactggacgaccgcctcccacttagcgaggctcggctgtgtcgtgttagagtctccctgtccggcccttcattcacactgaggcgttagaccgttctaataatcatcaaccctctcaatgttcagacctcctctctgacccaacttaaaactagacgagtttgaacgtccgctacatcttagtttaggacctgggcgggccctgtccaggtatcgagagtgcaaagagcgtcaacctcctcaagacgagaaggagaggcacttgcacgtgcgactgtgacccacacggtatctgtagtcggccgttctcgactctacaccttcacctcacaagtatg

SEQ ID NO: 5 - DNA, 2000 bp, Artificial Sequence
  West Nile Delta CprME - Rabies PIV sequence (partial)
agtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtctatttgctaaaacgcggaatgccccgcgtgttgtccttgattggacttaagcaaaagaagcgagggggcaagactggtatagctgtgatcgttcctcaggctcttttgtttgtacccttgctggtatttcccctttgctttggtaaatttcctatctataccatccctgataagctcgggccttggagtcccattgatattcaccatttgagctgcccaaacaacctcgtcgttgaggatgaagggtgcactaatctttctggattttcctacatggagttgaaagtgggctatatttcagccattaagatgaacggctttacttgtacaggagtcgtgaccgaagccgagacatatacaaatttcgtgggatacgtcaccaccaccttcaagagaaaacacttccgcccaacgcctgacgcttgtcgggccgcttacaactggaagatggcaggagatcctcgatatgaagaatctctgcacaacccgtatcctgattaccattggctgcggacagtcaagactaccaaggagagtctggtcattatatcaccaagcgtggccgatcttgatccttatgatagatccctgcacagtagggtttttcctggcgggaattgtagcggtgttgcagtatcaagtacctactgctccactaaccacgactacactatatggatgcctgagaaccctcgactcggtatgagttgcgacatttttacgaactcacggggcaagcgggcatctaaggggtctgaaacatgcgggtttgttgatgagcgggggttgtataaatctcttaaaggcgcctgtaagctgaaactctgtggcgtactggggctgcgcctgatggacggcacatgggtggctatgcagacaagcaatgaaacaaagtggtgtccccctggtcagctggttaatctgcacgactttaggtctgacgaaatcgagcaccttgtggtggaggaactggtgaagaaacgcgaagagtgcctggacgcacttgagagtattatgaccaccaaatccgtttccttcagaagactgagccacctgcgaaagctggtgccagggttcgggaaggcttatactattttcaacaagactcttatggaggcggatgcccattataagtcagttaggacttggaatgagataattccctccaaaggatgtctgagagtcggtgggagatgccacccccatgtcaatggggtgttctttaacggaatcatcctgggacctgacgggaacgtgctgattcccgagatgcaatcttcccttctgcagcaacacatggaactcctggtgtcttcagtgatacccctgatgcacccactggccgaccccagcactgtgttcaaaaatggcgatgaggccgaagactttgtggaagttcacctgcccgatgtacacgaaaggatatctggagtagacctgggccttcctaattggggtaagtacgtgctcctgagtgcgggtgccttgaccgctttgatgctgatcatttttctgatgacctgctggcggagggtgaatcgctccgagccgacacagcacaatctcagagggacaggccgggaagtaagtgtgactccgcaatctggcaagattattagtagttgggagagttacaagtctggaggagagactgggttgaattttgatctgctcaaacttgcaggcgatgtagaatcaaatcctggacccgcccgggacaggtccatagctctcacgtttctcgcagttggaggagttctgctcttcctctccgtgaacgtgcacgctgacactgggtgtgccatagacatcagccggcaagagctgagatgtggaagtggagtgttcatacacaatgatgt

SEQ ID NO: 6 - DNA, 2000 bp, Artificial Sequence
  West Nile Delta CprME - Rabies PIV sequence (partial) (complement)
tcatcaagcggacacactcgactgtttgaatcatcacaaacactcctaattgttgttaattgtgtcacgctcgacaaagaatcgtgcttctagagctacagattctttggtcctcccgggccgttctcggcccgacagataaacgattttgcgccttacggggcgcacaacaggaactaacctgaattcgttttcttcgctcccccgttctgaccatatcgacactagcaaggagtccgagaaaacaaacatgggaacgaccataaaggggaaacgaaaccatttaaaggatagatatggtagggactattcgagcccggaacctcagggtaactataagtggtaaactcgacgggtttgttggagcagcaactcctacttcccacgtgattagaaagacctaaaaggatgtacctcaactttcacccgatataaagtcggtaattctacttgccgaaatgaacatgtcctcagcactggcttcggctctgtatatgtttaaagcaccctatgcagtggtggtggaagttctcttttgtgaaggcgggttgcggactgcgaacagcccggcgaatgttgaccttctaccgtcctctaggagctatacttcttagagacgtgttgggcataggactaatggtaaccgacgcctgtcagttctgatggttcctctcagaccagtaatatagtggttcgcaccggctagaactaggaatactatctagggacgtgtcatcccaaaaaggaccgcccttaacatcgccacaacgtcatagttcatggatgacgaggtgattggtgctgatgtgatatacctacggactcttgggagctgagccatactcaacgctgtaaaaatgcttgagtgccccgttcgcccgtagattccccagactttgtacgcccaaacaactactcgcccccaacatatttagagaatttccgcggacattcgactttgagacaccgcatgaccccgacgcggactacctgccgtgtacccaccgatacgtctgttcgttactttgtttcaccacagggggaccagtcgaccaattagacgtgctgaaatccagactgctttagctcgtggaacaccacctccttgaccacttctttgcgcttctcacggacctgcgtgaactctcataatactggtggtttaggcaaaggaagtcttctgactcggtggacgctttcgaccacggtcccaagcccttccgaatatgataaaagttgttctgagaatacctccgcctacgggtaatattcagtcaatcctgaaccttactctattaagggaggtttcctacagactctcagccaccctctacggtgggggtacagttaccccacaagaaattgccttagtaggaccctggactgcccttgcacgactaagggctctacgttagaagggaagacgtcgttgtgtaccttgaggaccacagaagtcactatggggactacgtgggtgaccggctggggtcgtgacacaagtttttaccgctactccggcttctgaaacaccttcaagtggacgggctacatgtgctttcctatagacctcatctggacccggaaggattaaccccattcatgcacgaggactcacgcccacggaactggcgaaactacgactagtaaaaagactactggacgaccgcctcccacttagcgaggctcggctgtgtcgtgttagagtctccctgtccggcccttcattcacactgaggcgttagaccgttctaataatcatcaaccctctcaatgttcagacctcctctctgacccaacttaaaactagacgagtttgaacgtccgctacatcttagtttaggacctgggcgggccctgtccaggtatcgagagtgcaaagagcgtcaacctcctcaagacgagaaggagaggcacttgcacgtgcgactgtgacccacacggtatctgtagtcggccgttctcgactctacaccttcacctcacaagtatgtgttactaca

SEQ ID NO: 7 - DNA, 2700 bp, Artificial Sequence
  PIV-WNV helper Delta NS1
agtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtcaatatgctaaaacgcggaatgccccgcgtgttgtccttgattggacttaagagggctatgttgagcctgatcgacggcaaggggccaatacgatttgtgttggctctcttggcgttcttcaggttcacagcaattgctccgacccgagcagtgctggatcgatggagaggtgtgaacaaacaaacagcgatgaaacaccttctgagtttcaagaaggaactagggaccttgaccagtgctatcaatcggcggagctcaaaacaaaagaaaagaggaggaaagaccggaattgcagtcatgattggcctgatcgccagcgtaggagcagttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacttatgaatgcccagtgctgtcggctggtaatgatccagaagacatcgactgttggtgcacaaagtcagcagtctacgtcaggtatggaagatgcaccaagacacgccactcaagacgcagtcggaggtcactgacagtgcagacacacggagaaagcactctagcgaacaagaagggggcttggatggacagcaccaaggccacaaggtatttggtaaaaacagaatcatggatcttgaggaaccctggatatgccctggtggcagccgtcattggttggatgcttgggagcaacaccatgcagagagttgtgtttgtcgtgctattgcttttggtggccccagcttacagctttaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggccaacctggcagaggtccgcagttattgctatttggctaccgtcagcgatctctccaccaaagctgcgtgcccggccatgggagaagctcacaatgacaaacgtgctgacccagcttttgtgtgcagacaaggagtggtggacaggggctggggcaacggctgcggactatttggcaaaggaagcattgacacatgcgccaaatttgcctgctctaccaaggcaataggaagaaccattttgaaagagaatatcaagtacgaagtggccatttttgtccatggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagtggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccactggcacaagtctggaagcagcattggcaaagcctttacaaccaccctcaaaggagcgcagagactagccgctctaggagacacagcttgggactttggatcagttggaggggtgttcacctcagttgggaaggctgtccatcaagtgttcggaggagcattccgctcactgttcggaggcatgtcctggataacgcaaggattgctgggggctctcctgttgtggatgggcatcaatgctcgtgacaggtccatagctctcacgtttctcgcagttggaggagttctgctcttcctctccgtgaacgtgcacgctgacactgggatccaccgtggacctgccactcgcaccaccacagagagcggaaagttgataacagattggtgctgcaggagctgcaccttaccaccactgcgctaccaaactgacagcggctgttggtatggtatggagatcagaccacagagacatgatgaaaagaccctcgtgcagtcacaagtgaatgcttataatgctgatatgattgacccttttcagttgggcctt

SEQ ID NO: 8 - DNA, 2700 bp, Artificial Sequence
  PIV-WNV helper Delta NS1 (complement)
tcatcaagcggacacactcgactgtttgaatcatcacaaacactcctaattgttgttaattgtgtcacgctcgacaaagaatcgtgcttctagagctacagattctttggtcctcccgggccgttctcggcccgacagttatacgattttgcgccttacggggcgcacaacaggaactaacctgaattctcccgatacaactcggactagctgccgttccccggttatgctaaacacaaccgagagaaccgcaagaagtccaagtgtcgttaacgaggctgggctcgtcacgacctagctacctctccacacttgtttgtttgtcgctactttgtggaagactcaaagttcttccttgatccctggaactggtcacgatagttagccgcctcgagttttgttttcttttctcctcctttctggccttaacgtcagtactaaccggactagcggtcgcatcctcgtcaatgggagagattgaaggttcccttccactactactgccatttacgatgactgcagtgtctacagtagtgctaaggttgtcgacgacctttcttggatacgtaacagtctcgttacctacaccctatgtacacgctactatgatagtgaatacttacgggtcacgacagccgaccattactaggtcttctgtagctgacaaccacgtgtttcagtcgtcagatgcagtccataccttctacgtggttctgtgcggtgagttctgcgtcagcctccagtgactgtcacgtctgtgtgcctctttcgtgagatcgcttgttcttcccccgaacctacctgtcgtggttccggtgttccataaaccatttttgtcttagtacctagaactccttgggacctatacgggaccaccgtcggcagtaaccaacctacgaaccctcgttgtggtacgtctctcaacacaaacagcacgataacgaaaaccaccggggtcgaatgtcgaaattgacggaaccttactcgttgtctctgaagaaccttcctcacagacctcgttgtacccacctaaaccaagagcttccgctgtcgacgcactgatagtacagattcctgttcggatggtagctacacttctactacttatacctccgccggttggaccgtctccaggcgtcaataacgataaaccgatggcagtcgctagagaggtggtttcgacgcacgggccggtaccctcttcgagtgttactgtttgcacgactgggtcgaaaacacacgtctgttcctcaccacctgtccccgaccccgttgccgacgcctgataaaccgtttccttcgtaactgtgtacgcggtttaaacggacgagatggttccgttatccttcttggtaaaactttctcttatagttcatgcttcaccggtaaaaacaggtacctggttgatgacacctcagcgtgcctttgatgaggtgtgtccaacctcggtgagtccgtccctctaagtcgtagtgaggacgccgcggaagtatgtgtgatttcgaacctcttatacctctccactgtcacctgacacttggtgccagtccctaactgtggttacgtatgatgcactactgacaaccttgtttctgcaagaaccaggtagcactcaccaagtacctggagttggagggaacctcgtcacgaccttcatgacacacctccttgtctctctgcaattacctcaaactccttggtgtgcggtgcttcgtcagacactatcgtaacccgagtgttctccctcgagacgtagttcgaaaccgacctcggtaaggacaccttaaaagttcgttgtgacagttcaactgcagcccagtaaacttcacatctcacttctacctttttaacgtcaacttcccttgttggataccgcagacaagtttccgaaagttcaaagaaccctgagggcgtctgtgtccagtgccgtgacaccacaaccttaacgtcatgtgaccgtgcctacctggaacgtttcaaggatagagcagtcaccgaagtaacttgctggattgcggtcacccgtctaaccagtgacagttgggaaaacaaagtcaccggtgccggttgcgattccaggactaacttaaccttggtgggaaacctctgagtatgtatcaccacccgtctcctcttgttgtctagttagtggtgaccgtgttcagaccttcgtcgtaaccgtttcggaaatgttggtgggagtttcctcgcgtctctgatcggcgagatcctctgtgtcgaaccctgaaacctagtcaacctccccacaagtggagtcaacccttccgacaggtagttcacaagcctcctcgtaaggcgagtgacaagcctccgtacaggacctattgcgttcctaacgacccccgagaggacaacacctacccgtagttacgagcactgtccaggtatcgagagtgcaaagagcgtcaacctcctcaagacgagaaggagaggcacttgcacgtgcgactgtgaccctaggtggcacctggacggtgagcgtggtggtgtctctcgcctttcaactattgtctaaccacgacgtcctcgacgtggaatggtggtgacgcgatggtttgactgtcgccgacaaccataccatacctctagtctggtgtctctgtactacttttctgggagcacgtcagtgttcacttacgaatattacgactatactaactgggaaaagtcaacccggaa

SEQ ID NO: 9 - DNA, 1572 bp, Artificial Sequence
  Rabies G from NC_001542
atggttcctcaggctctcctgtttgtaccccttctggtttttccattgtgttttgggaaattccctatttacacgataccagacaagcttggtccctggagcccgattgacatacatcacctcagctgcccaaacaatttggtagtggaggacgaaggatgcaccaacctgtcagggttctcctacatggaacttaaagttggatacatctcagccataaaaatgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccactggcttcgaactgtaaaaaccaccaaggagtctctcgttatcatatctccaagtgtggcagatttggacccatatgacagatcccttcactcgagggtcttccctggcgggaattgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtcgcgatgcaaacatcaaatgaaaccaaatggtgccctcccggtcagttggtgaatttgcacgactttcgctcagacgaaattgagcaccttgttgtagaggagttggtcaagaagagagaggagtgtctggatgcactagagtccatcatgaccaccaagtcagtgagtttcagacgtctcagtcatttaagaaaacttgtccctgggtttggaaaagcatataccatattcaacaagaccttgatggaagccgatgctcactacaagtcagtcagaacttggaatgagatcatcccttcaaaagggtgtttaagagttggggggaggtgtcatcctcatgtaaacggggtatttttcaatggtataatattaggacctgacggcaatgtcttaatcccagagatgcaatcatccctcctccagcaacatatggagttgttggtatcctcggttatcccccttatgcaccccctggcagacccgtctaccgttttcaagaacggtgacgaggctgaggattttgttgaagttcaccttcccgatgtgcacgaacggatctcaggagttgacttgggtctcccgaactgggggaagtatgtattactgagtgcaggggccctgactgccttgatgttgataattttcctgatgacatgctggagaagagtcaatcgatcggaacctacacaacacaatctcagagggacagggagggaggtgtcagtcactccccaaagcgggaagatcatatcttcatgggaatcatacaagagcgggggtgagaccggactg

SEQ ID NO: 10 - protein, 524 aa, Artificial Sequence
  Rabies G from NC_001542
MVPQALLFVPLLVFPLCFGKFPIYTIPDKLGPWSPIDIHHLSCPNNLVVEDEGCTNLSGFSYMELKVGYISAIKMNGFTCTGVVTEAETYTNFVGYVTTTFKRKHFRPTPDACRAAYNWKMAGDPRYEESLHNPYPDYHWLRTVKTTKESLVIISPSVADLDPYDRSLHSRVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHLPDVHERISGVDLGLPNWGKYVLLSAGALTALMLIIFLMTCWRRVNRSEPTQHNLRGTGREVSVTPQSGKIISSWESYKSGGETGL

SEQ ID NO: 11 - DNA, 1426 bp, Artificial Sequence
  Codon-optimized Rabies G
atcgttcctcaggctcttttgtttgtacccttgctggtatttcccctttgctttggtaaatttcctatctataccatccctgataagctcgggccttggagtcccattgatattcaccatttgagctgcccaaacaacctcgtcgttgaggatgaagggtgcactaatctttctggattttcctacatggagttgaaagtgggctatatttcagccattaagatgaacggctttacttgtacaggagtcgtgaccgaagccgagacatatacaaatttcgtgggatacgtcaccaccaccttcaagagaaaacacttccgcccaacgcctgacgcttgtcgggccgcttacaactggaagatggcaggagatcctcgatatgaagaatctctgcacaacccgtatcctgattaccattggctgcggacagtcaagactaccaaggagagtctggtcattatatcaccaagcgtggccgatcttgatccttatgatagatccctgcacagtagggtttttcctggcgggaattgtagcggtgttgcagtatcaagtacctactgctccactaaccacgactacactatatggatgcctgagaaccctcgactcggtatgagttgcgacatttttacgaactcacggggcaagcgggcatctaaggggtctgaaacatgcgggtttgttgatgagcgggggttgtataaatctcttaaaggcgcctgtaagctgaaactctgtggcgtactggggctgcgcctgatggacggcacatgggtggctatgcagacaagcaatgaaacaaagtggtgtccccctggtcagctggttaatctgcacgactttaggtctgacgaaatcgagcaccttgtggtggaggaactggtgaagaaacgcgaagagtgcctggacgcacttgagagtattatgaccaccaaatccgtttccttcagaagactgagccacctgcgaaagctggtgccagggttcgggaaggcttatactattttcaacaagactcttatggaggcggatgcccattataagtcagttaggacttggaatgagataattccctccaaaggatgtctgagagtcggtgggagatgccacccccatgtcaatggggtgttctttaacggaatcatcctgggacctgacgggaacgtgctgattcccgagatgcaatcttcccttctgcagcaacacatggaactcctggtgtcttcagtgatacccctgatgcacccactggccgaccccagcactgtgttcaaaaatggcgatgaggccgaagactttgtggaagttcacctgcccgatgtacacgaaaggatatctggagtagacctgggccttcctaattggggtaagtacgtgctcctgagtgcgggtgccttgaccgctttgatgctgatcatttttc

SEQ ID NO: 12 - protein, 524 aa, Artificial Sequence
  Codon-optimized Rabies G (begins with “I” instead of “M” in the constructs)
IVPQALLFVPLLVFPLCFGKFPIYTIPDKLGPWSPIDIHHLSCPNNLVVEDEGCTNLSGFSYMELKVGYISAIKMNGFTCTGVVTEAETYTNFVGYVTTTFKRKHFRPTPDACRAAYNWKMAGDPRYEESLHNPYPDYHWLRTVKTTKESLVIISPSVADLDPYDRSLHSRVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHLPDVHERISGVDLGLPNWGKYVLLSAGALTALMLIIFLMTCWRRVNRSEPTQHNLRGTGREVSVTPQSGKIISSWESYKSGGETGL

(19) United States
(12) Patent Application Publication
(10) Pub. No.: US 2014/0271708 A1
(43) Pub. Date: Sep. 18, 2014

(54) Novel methods for providing long-term protective immunity against rabies in animals, based upon administration of replication-deficient flavivirus expressing rabies G

(71) Applicant: Frederic David, Watkinsville, GA (US)

(72) Inventor: Frederic David, Watkinsville, GA (US)

(73) Assignee: MERIAL LIMITED, Duluth, GA (US)

(21) Appl. No.: 13/841,383

(22) Filed: Mar. 15, 2013

(51) Int. Cl. A61K 39/205

(52) U.S. Cl. CPC A61K 39/205
USPC 424/199.1

(57) ABSTRACT

The present invention relates to compositions comprising replication defective chimeric flavivirus anti-rabies vaccines, methods of producing the vaccines, and the administration of such vaccines to companion animals, including dogs. The invention further relates to methods for providing long-term protective immunity against rabies in companion animals, including dogs and cats.

### INCORPORATION BY REFERENCE

This application claims priority to U.S. provisional patent application No. 61/611,604, which was filed on Mar. 16, 2012, and is herein incorporated by reference in its entirety.

### FIELD OF THE INVENTION

The present invention relates to non-adjuvanted, recombinant rabies vaccines having strong safety and efficacy profiles.

### BACKGROUND

Rabies virus is a non-segmented negative-stranded RNA virus of the Rhabdoviridae family. Rabies virus virions are composed of two major structural components: a nucleocapsid or ribonucleoprotein (RNP), and an envelope in the form of a bilayer membrane surrounding the RNP core. The infectious component of all Rhabdoviruses is the RNP core which consists of the RNA genome encapsidated by the nucleocapsid (N) protein in combination with two minor proteins, i.e. RNA-dependent RNA-polymerase (L) and phosphoprotein (P). The membrane surrounding the RNP core consists of two proteins: a trans-membrane glycoprotein (G) and a matrix (M) protein located at the inner site of the membrane. The G protein, also referred to as spike protein, is responsible for cell attachment and membrane fusion in rabies virus and additionally is the main target for the host immune system. The amino acid region at position 330 to 340 (referred to as antigenic site III) of the G protein has been identified to be responsible for the virulence of the virus, in particular the Arg residue at position 333. All rabies virus strains have this virulence determining antigenic site III in common.

With few exceptions, rabies invariably results in fatal neurological disease in humans and animals, and remains a serious global public health concern. The majority of human deaths stemming from rabies have occurred in Africa, Asia and South America but a rabies epidemic has also recently become problematic in the United States due to a rapidly growing population of infected raccoons. Other primary virus carriers of concern are the skunk, largely in the mid-western states, and bats, the main source for most human cases in the U.S. In addition to the infected wildlife such as the raccoons, skunks, foxes, wolves, etc., humans typically become infected with rabies through the bite of infected dogs and cats. Dogs continue to be the main hosts of the rabies virus in Africa and Asia where canine rabies is endemic and are still responsible for most of the human deaths that occur from rabies worldwide. It is of particular importance to mankind, therefore, to prevent the rabies virus infection in domestic pets such as dogs, cats and ferrets.

In view of the disadvantages of conventional rabies viral vaccines, research has been aimed at the use of recombinant vaccines. For example, the Oral Rabies Vaccination (ORV) program, which deploys vaccinia-vectored recombinant Rabies vaccine (RABORAL V-RG®, Merial Limited) has had a positive effect in reducing the frequency of rabies in US wildlife populations. Other poxviruses have also been applied to the problem of producing safe, effective rabies vaccines. For example, raccoon pox (disclosed in US2005/0282210, to Merial Limited).

Prior to the instant disclosure, inventors are unaware of anyone successfully using the REPLIVAX flavivirus-based system to express Rabies G and elicit protective immunity in canine against rabies. The vectors used herein to produce surprising, unpredictable, and long-lasted protective immunity in canine animals are described in US 2011/0135686 A1 (to Sanofi Pasteur, the disclosure of which is herein incorporated by reference in its entirety). A key element of REPLIVAX is PIV technology, principles of which are illustrated in FIGS. 1A and 1B. There are two variations of the technology. In the first variation, a single-component pseudoinfectious virus (s-PIV) is constructed with a large deletion in the capsid protein (C), rendering mutant virus unable to form infectious viral particles in normal cells (FIG. 1B). The deletion does not remove the first˜20 codons of the C protein, which contain an RNA cyclization sequence, and a similar number of codons at the end of C, which encode a viral protease cleavage site and the signal peptide for prM. The s-PIV can be propagated, e.g., during manufacture, in substrate (helper) cell cultures in which the C protein is supplied in trans, e.g., in stably transfected cells producing the C protein (or a larger helper cassette including C protein), or in cells containing an alphavirus replicon [e.g., a Venezuelan equine encephalitis virus (VEE) replicon] expressing the C protein or another intracellular expression vector expressing the C protein. Following inoculation in vivo, e.g., after immunization, the PIV undergoes a single round of replication in infected cells in the absence of trans-complementation of the deletion, without spread to surrounding cells. The infected cells produce empty virus-like particles (VLPs), which are the product of the prM-E genes in the PIV, resulting in the induction of neutralizing antibody response. A T-cell response should also be induced via MHCI presentation of viral epitopes. This approach has been applied to YF 17D virus and WN viruses and WN/JE and WN/DEN2 chimeric viruses (Mason et al., Virology 351:432-443, 2006; Suzuki et al., J. Virol. 83: 1870-1880, 2009; Ishikawa et al., Vaccine 26:2772-2781, 2008; Widman et al., Vaccine 26:2762-2771, 2008; WO 2007/098267; WO 2008/137163).

In the second variation, a two-component PIV (d-PIV) is constructed. Substrate cells are transfected with two defective viral RNAs, one with a deletion in the C gene and another lacking the prM-E envelope protein genes. The two defective genomes complement each other, resulting in accumulation of two types of PIVs in the cell culture medium (Shustov et al., J. Virol. 21 :1 1737-1 1748, 2007; Suzuki et al., J. Virol. 82:6942-6951, 2008). Optionally, the two PIVs can be manufactured separately in appropriate helper cell lines and then mixed in a two-component formulation. The latter may offer an advantage of adjusting relative concentrations of the two components, increasing immunogenicity and efficacy. This type of PIV vaccine should be able to undergo a limited spread in vivo due to co-infection of some cells at the site of inoculation with both components. The spread is expected to be self-limiting as there are more cells in tissues than viral particles produced by initially co-infected cells. In addition, a relatively high MOI is necessary for efficient co-infection, and cells outside of the inoculation site are not expected to be efficiently co-infected (e.g., in draining lymph nodes). Cells infected with the AC PIV alone produce the highly immunogenic VLPs. Co-infected cells produce the two types of packaged defective viral particles, which also stimulate neutralizing antibodies. The limited infection is expected to result in a stronger neutralizing antibody response and T-cell response compared to s-PIVs. To decrease chances of recombination during manufacture or in vivo, including with circulating flaviviruses, viral sequences can be modified in both s-PIVs and d-PIVs using, e.g., synonymous codon replacements, to reduce nucleotide sequence homologies, and mutating the complementary cyclization 5′ and 3′ elements.

As the REPLIVAX system is clearly quite powerful, and addresses long-felt needs in the vaccine field, it is the object of this disclosure to provide compositions and methods for providing protective immunity in animals, including canine animals, against rabies using modified REPLIVAX flavivirus-based constructs, containing and expressing in vivo non-flavivirus genes, particularly wildtype and mutant Rabies G genes.

### SUMMARY OF THE INVENTION

The invention is based, in part, on the unexpected and surprising result that a non-flavivirus antigen, the Rabies G protein (RabG), expressed from a replication-defective flavivirus vector, RepliVax® is sufficient to confer protective immunity in an animal, including a canine animal, against rabies.

The invention provides several Replivax® West Nile (WN) vectors, which have been modified to contain and express in vivo the RabG gene (as described in US 2011/0135686 A1, to Sanofi Pasteur). In an embodiment, the construct comprises Rabies G, prM, E, and NS; C, Rabies G, NS; or Rabies G (AC230G) and NS. The resulting RepliVax®-RabG (RV-RabG) constructs can be propagated as single-component vaccines in appropriate helper cell lines, or as two-component vaccines in naïve cells. Inventors herein provide evidence that the single-component RV-RabG variants replicate to high titers (8 logs) in complementing helper cells, and do not spread to normal, non-complementing, cells (and especially the cells of the vaccinate). The vectors are capable of robust RabG expression, maintain full-length insert through multiple rounds of in vitro passaging, and produce durable immune responses. In an embodiment, the invention provides a safe, avirulent, two-component vaccine variant.

The invention also provides dual vaccines, capable of inducing immunogenic responses against both rabies and WN, and kits for performing any of the above described methods comprising the any of the above described compositions and optionally, instructions for performing the method. No interference was observed between co-administered RepliVax® constructs.

Another embodiment of the present invention provides for a stable, safe and easily administrable vaccine. In an embodiment, the vaccine may be wildlife bait that is consumed by animals including, but not solely, raccoons, coyotes, foxes, rabbits, bats, squirrels, canines, and felines.

These and other embodiments are disclosed or are obvious from and encompassed by, the following Detailed Description.

### BRIEF DESCRIPTION OF DRAWINGS

A full and enabling disclosure of the present invention, including the best mode thereof, to one of ordinary skill in the art, is set forth more particularly in the remainder of the specification, including reference to the accompanying figures, wherein:

FIG. 1A provides a schematic of construction of the RepliVax constructs. As illustrated, single-component RepliVax (or pseudo infectious virus, sPIV) is made by introducing a deletion removing most of the C protein gene, with the exception of the first 20 codons containing the RNA cyclization signal necessary for replication. The two-component RepliVax (tcPIV) is composed of two trans-complementing genomes, one with the ΔC deletion (as in sPIV), and the other with deletion of the prM-E genes;

FIG. 1B depicts sPIV RepliVax propagation in packaging helper cells, which supply the complete C protein in trans (or optionally the C-prM-E cassette);

FIG. 2 presents diagrams of constructs used to produce pseudoinfectious viruses in BHK packaging cell line containing the rVEE helper replicon encoding West Nile genes C, prM, and E;

FIG. 3 presents schematic representation for obtaining the inventive PIV;

FIG. 4 presents RFFIT titers for the mouse vaccination study;

FIG. 5A is a line plot of antibody titers by RFFIT (IU/ml) for Dogs vaccinated with AprME RabG 230G;

FIG. 5B is a line plot of antibody titers for high and low dose Dog groups challenged on days 378 and 757, respectively;

FIG. 6 is a line plot of antibody titers for Pigs vaccinated with high (group A) and low (group B) doses of AprME RabG 230G.

### DETAILED DESCRIPTION OF THE INVENTION

Other objects, features and aspects of the present invention are disclosed in, or are obvious from, the following Detailed Description. It is to be understood by one of ordinary skill in the art that the present discussion is a description of exemplary embodiments only and is not intended as limiting the broader aspects of the present invention, which broader aspects are embodied in the exemplary construction. In fact, it will be apparent to those skilled in the art that various modifications and variations can be made in the present invention without departing from the scope or spirit of the invention. For instance, features illustrated or described as part of one embodiment can be used in another embodiment to yield a still further embodiment. It is intended that the present invention cover such modifications and variations as come within the scope of the appended claims and their equivalents. The contents of all references, published patents, and patents cited throughout the present application are hereby incorporated by reference in their entirety.

In a first aspect, the present invention provides a RepliVax system of viral constructs modified to express non-flavivirus genes. In a particular embodiment, the genes are wild type or mutant rabies G protein.

In a second aspect, the present invention provides methods of constructing said viral constructs.

In a third aspect, the invention provides for immunogenic or vaccine compositions comprising one or more viral constructs. In an embodiment, the constructs are transduced into helper cells expressing one or more flavivirus genes, necessary for the assembly of construct-encoded gene products into pseudoinfectious viral particles. In a particular embodiment the constructs comprise Rabies G+WNV (i.e. flaviviral) genes according to the following: 1) Rabies G, prM, E, and NS; C, Rabies G, NS; or Rabies G (AC230G) and NS.

In a fourth aspect, the present invention provides for immunogenic or vaccine compositions comprising pseudoinfectious virus particles comprising non-flavivirus antigens. In a particular embodiment, the antigens are rabies G or rabies G 230G.

In a fifth aspect, the present invent provides for methods of producing the immunogenic or vaccine compositions.

In a sixth aspect, the present invention provides for methods preventing disease in an animal, comprising the steps of administering the immunogenic or vaccine compositions according to the instant disclosure.

For convenience, certain terms employed in the Specification, Examples, and appended Claims are collected here.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicate otherwise.

It is also noted that in this disclosure and particularly in the claims, terms such as “comprises”, “comprised”, “comprising” and the like can have the meaning attributed to such terms in U.S. patent law; e.g., they can mean “includes”, “included”, “including”, and the like; and that terms such as “consisting essentially of” and “consists essentially of” have the meaning ascribed to them by U.S. patent law, e.g., they allow for elements not explicitly recited, but exclude elements that are found in the prior art or that affect a basic or novel characteristic of the invention.

As used herein, the term “animal” includes all vertebrate animals including humans. Animal or host includes mammals and human. The animal may be selected from the group consisting of equine (e.g., horse), canine (e.g., dogs, wolves, foxes, coyotes, jackals), feline (e.g., lions, tigers, domestic cats, wild cats, other big cats, and other felines including cheetahs and lynx), ovine (e.g., sheep), bovine (e.g., cattle), porcine (e.g., pig), caprine (e.g., goat), avian (e.g., chicken, duck, goose, turkey, quail, pheasant, parrot, finches, hawk, crow, ostrich, emu and cassowary), primate (e.g., prosimian, tarsier, monkey, gibbon, ape), and fish. The term “animal” also includes an individual animal in all stages of development, including embryonic and fetal stages.

As used herein, the term “virulent” means an isolate that retains its ability to be infectious in an animal host.

As used herein, the term “inactivated vaccine” means a vaccine composition containing an infectious organism or pathogen that is no longer capable of replication or growth. The pathogen may be bacterial, viral, protozoal or fungal in origin. Inactivation may be accomplished by a variety of methods including freeze-thawing, chemical treatment (for example, treatment with formalin), sonication, radiation, heat or any other convention means sufficient to prevent replication or growth of the organism while maintaining its immunogenicity.

As used herein, the term “immunogenicity” means capable of producing an immune response in a host animal against an antigen or antigens. This immune response forms the basis of the protective immunity elicited by a vaccine against a specific infectious organism.

As used herein, the term “immune response” refers to a response elicited in an animal. An immune response may refer to cellular immunity (CMI); humoral immunity or may involve both. The present invention also contemplates a response limited to a part of the immune system. For example, a vaccine composition of the present invention may specifically induce an increased gamma interferon response.

As used herein, the term “antigen” or “immunogen” means a substance that induces a specific immune response in a host animal. The antigen may comprise a whole organism, killed, attenuated or live; a subunit or portion of an organism; a recombinant vector containing an insert with immunogenic properties; a piece or fragment of DNA capable of inducing an immune response upon presentation to a host animal; a protein, a polypeptide, a peptide, an epitope, a hapten, or any combination thereof. Alternately, the immunogen or antigen may comprise a toxin or antitoxin.

As used herein, the term “multivalent” means a vaccine containing more than one antigen whether from the same species (i.e., different isolates of Rabies virus serotypes), from a different species (i.e., isolates from both canine distemper and canine parvovirus), or a vaccine containing a combination of antigens from different genera (for example, a vaccine comprising antigens from leptospira spp., rabies, lyme disease, west nile virus and parainfluenza).

As used herein, the term “adjuvant” means a substance added to a vaccine to increase a vaccine's immunogenicity, as compared with its efficacy in absence of the adjuvant. The mechanism of how adjuvants operate is not entirely known. Some adjuvants are believed to enhance the immune response by slowly releasing the antigen, while other adjuvants are strongly immunogenic in their own right and are believed to function synergistically. Known vaccine adjuvants include, but are not limited to, oil and water emulsions (for example, complete Freund's adjuvant and incomplete Freund's adjuvant, and adjuvants disclosed in U.S. Pat. No. 7,371,395 to Merial Limited, which are herein incorporated by reference in their entirety), Corynebacterium parvum, Bacillus Calmette Guerin, aluminum hydroxide, glucan, dextran sulfate, iron oxide, sodium alginate, Bacto-Adjuvant, certain synthetic polymers such as poly amino acids and co-polymers of amino acids, saponin, “REGRESSIN” (Vetrepharm, Athens, Ga.), “AVRIDINE” (N,N-dioctadecyl-N′,N′-bis(2-hydroxyethyl)-propanediamine), paraffin oil, muramyl dipeptide and the like.

As used herein, the term “emulsion” refers to a combination of at least two substances, wherein a first substance is dispersed in a second substance in which the first substance is insoluble. One example of an emulsion of the present invention is an oil phase dispersed in an aqueous phase.

As used herein, the terms “pharmaceutically acceptable carrier” and “pharmaceutically acceptable vehicle” are interchangeable and refer to a fluid vehicle for containing vaccine antigens that can be injected into a host without adverse effects. Suitable pharmaceutically acceptable carriers known in the art include, but are not limited to, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, colors and the like.

As used herein, the term “vaccine composition” includes at least one antigen or immunogen in a pharmaceutically acceptable vehicle useful for inducing an immune response in a host. Vaccine compositions can be administered in dosages and by techniques well known to those skilled in the medical or veterinary arts, taking into consideration such factors as the age, sex, weight, species and condition of the recipient animal, and the route of administration. The route of administration can be percutaneous, via mucosal administration (e.g., oral, nasal, anal, vaginal) or via a parenteral route (intradermal, intramuscular, subcutaneous, intravenous, or intraperitoneal). Vaccine compositions can be administered alone, or can be co-administered or sequentially administered with other treatments or therapies. Forms of administration may include suspensions, syrups or elixirs, and preparations for parenteral, subcutaneous, intradermal, intramuscular or intravenous administration (e.g., injectable administration) such as sterile suspensions or emulsions. Vaccine compositions may be administered as a spray or mixed in food and/or water or delivered in admixture with a suitable carrier, diluent, or excipient such as sterile water, physiological saline, glucose, or the like. The compositions can contain auxiliary substances such as wetting or emulsifying agents, pH buffering agents, adjuvants, gelling or viscosity enhancing additives, preservatives, flavoring agents, colors, and the like, depending upon the route of administration and the preparation desired. Standard pharmaceutical texts, such as “Remington's Pharmaceutical Sciences,” 1990 may be consulted to prepare suitable preparations, without undue experimentation.

The term “purified” as used herein does not require absolute purity; rather, it is intended as a relative term. Thus, for example, a purified immunogen preparation, such as protein or inactivated virus, is one in which the immunogen is more enriched than the immunogen is in its natural environment. An immunogen preparation is herein broadly referred to as “purified” such that the immunogen represents at least 60%, at least 70%, at least 80%, at least 90%, at least 95%, or at least 98%, of the total immunogen content of the preparation. A “crude preparation”, which represents the lowest degree of purification, may contain as little as less than 60%, lest than 20%, less than 10%, less than 5%, or less than 1% of immunogenic components.

The term “highly purified” as used herein is intended to suggest a “higher degree of purity” as compared to the term “moderately purified”. This “higher degree of purity” can include, but is in no way limited to, reduced percentages of contaminants, in an immunological preparation that has been “highly purified” versus an immunological preparation that has been “moderately purified”. As discussed herein, “highly purified” immunological preparations will have the lowest to undetectable percentages of contaminants that can cause: reduced desired immune response, increased undesired immune response (e.g. hypersensitivity reaction), or reduced formulation stability. Similarly, an immunological preparation that has been “moderately purified” contains relatively reduced percentages of contaminants versus an immunological preparation that has been “minimally purified”, which likewise, has reduced percentages of contaminants versus a preparation designated a “crude preparation”.

Contaminants in an immunological preparation can include, but are in no way limited to, substances that contribute negatively to an immunological composition according to the present invention. One of several examples of a contaminant contributing negatively would be a contaminant that reduces the ability of an immunological composition of the present invention to elicit an immune response in animals.

Varying levels of purity (e.g. “highly purified”, “moderately purified”, and the like) can be achieved using various methods. For example, a combination of chromatography and size exclusion gel filtration can result in a “highly purified” or “moderately purified” immunological preparations. Differences in source/type of immunogens, as well as slight variations in purification procedures can significantly affect the final degree of immunogen purity. In general, as used herein, immunological preparations having the lowest to highest percentage of contaminants will be described as 1) “highly purified, 2) “moderately purified”, 3) “minimally purified”, 4) “crude preparation”, respectively. A “highly purified” preparation will have the lowest level across all types of contaminants. A “moderately purified” preparation will have relatively low levels of most types of contaminants, but may have one type of contaminant in higher abundance than would be observed for a comparable “highly purified” preparation. Likewise, a “minimally purified preparation” will have relatively low levels of some types of contaminants, but may have more than one type of contaminant in higher abundance than a comparable “moderately purified” preparation. As expected, a “crude preparation” has the highest level of contaminants, across all contaminant types, as compared to the other types of preparations discussed herein.

In another embodiment, the rabies glycoprotein is any rabies glycoprotein with a known protein sequence, such as rabies virus glycoprotein G. such as the protein sequences in or derived from the nucleotide sequences in Marissen et al., J. Virol. April 2005; 79(8):4672-8; Dietzschold et al., Vaccine. Dec. 9, 2004; 23(4):518-24; Mansfield et al., J Gen Virol. November 2004; 85(Pt 11):3279-83; Sato et al., J Vet Med Sci. July 2004; 66(7):747-53; Takayama-Ito et al., J. Neurovirol. April 2004; 10(2):131-5; Li et al., Zhongguo Yi Xue Ke Xue Yuan Xue Bao. December 2003; 25(6):650-4; Hemachudha et al., J Infect Dis. Oct. 1, 2003; 188(7):960-6; Kankanamge et al., Microbiol Immunol. 2003; 47(7):507-19; Maillard et al., Virus Res. June 2003; 93(2):151-8; Irie et al. Microbiol Immunol. 2002; 46(7):449-61; Langevin et al., J Biol Chem. Oct. 4, 2002; 277(40):37655-62; Maillard and Gaudin, J Gen Virol. June 2002; 83(Pt 6):1465-76; Holmes et al., Virology. Jan. 20, 2002; 292(2):247-57; Mebatsion, J. Virol. December 2001; 75(23):11496-502; Zhang et al., Zhonghua Shi Yan He Lin Chuang Bing Du Xue Za Zhi. September 2000; 14(3):281-4; Ray et al., Clin Exp Immunol. July 2001; 125(1):94-101; Morimoto et al., Vaccine. May 14, 2001; 19(25-26):3543-51; Morimoto et al., J. Neurovirol. October 2000; 6(5):373-81; Bourhy et al., J Gen Virol. October 1999; 80 (Pt 10):2545-57; Kissi et al., J Gen Virol. August 1999; 80 (Pt 8):2041-50; Nakahara et al., Microbiol Immunol. 1999; 43(3):259-70; Matthews et al., J Gen Virol. February 1999; 80 (Pt 2):345-53; Tuffereau et al., EMBO J. Dec. 15, 1998; 17(24):7250-9; Janet et al. J. Virol. January 1999; 73(1):225-33; Wloch et al., Hum Gene Ther. Jul. 1, 1998; 9(10):1439-47; Mellquist et al., Biochemistry. May 12, 1998; 37(19):6833-7; Morimoto et al., Proc Natl Acad Sci USA. Mar. 17, 1998; 95(6):3152-6; Coll, Arch Virol. 1997; 142(10):2089-97; Bracci et al., Blood. Nov. 1, 1997; 90(9):3623-8; Gaudin et al., J. Virol. November 1996; 70(11):7371-8; Morimoto et al., Proc Natl Acad Sci USA. May 28, 1996; 93(11):5653-8; Mebatsion et al., Cell. Mar. 22, 1996; 84(6):941-51; Shakin-Eshleman et al., J Biol Chem. Mar. 15, 1996; 271(11):6363-6; Nadin-Davis et al., J Virol Methods. March 1996; 57(1):1-14. Erratum in: J Virol Methods Apr. 26, 1996; 58(1-2):209; Wojczyk et al., Protein Exp. Purif. March 1996; 7(2):183-93; Suzuki et al., J Gen Virol. December 1995; 76 (Pt 12):3021-9; Raux et al., Virology. Jul. 10, 1995; 210(2):400-8; Kasturi et al., J Biol Chem. Jun. 16, 1995; 270(24):14756-61; Otvos et al., Biochim Biophys Acta. May 29, 1995; 1267(1):55-64; Mebatsion et al., J. Virol. March 1995; 69(3):1444-51; Wojczyk B, Shakin-Eshleman S H, Doms R W, Xiang Z Q, Ertl H C, Wunner W H, Spitalnik, Biochemistry. Feb. 28, 1995; 34(8):2599-609; Ravkov et al., Virology. Jan. 10, 1995; 206(1):718-23; Ni et al., Microbiol Immunol. 1995; 39(9):693-702; Coll, Arch Virol. 1995; 140(5):827-51; Grabko et al., Dokl Akad Nauk. July 1994; 337(1):117-21; Sakamoto et al., Virus Genes. January 1994; 8(1):35-46; Fodor et al., Arch Virol. 1994; 135(3-4):451-9; Ito et al., Microbiol Immunol. 1994; 38(6):479-82; Shakin-Eshleman et al., Biochemistry. Sep. 14, 1993; 32(36):9465-72; Morimoto et al., Virology. August 1993; 195(2):541-9; van der Heijden et al., J Gen Virol. August 1993; 74 (Pt 8):1539-45; Nishihara et al., Gene. Jul. 30, 1993; 129(2):207-14; Rustici et al., Biopolymers. June 1993; 33(6):961-9; McColl et al., Aust Vet J. March 1993; 70(3):84-9; Bai et al., Virus Res. February 1993; 27(2):101-12; Nishihara et al., Nippon Rinsho. February 1993; 51(2):323-8; Bracci et al., FEBS Lett. Oct. 19, 1992; 311(2):115-8; Tuchiya et al., Virus Res. Sep. 1, 1992; 25(1-2):1-13; Shakin-Eshleman et al., J Biol Chem. May 25, 1992; 267(15):10690-8; Whitt et al., Virology. December 1991; 185(2):681-8; Benmansour et al., J. Virol. August 1991; 65(8):4198-203; Burger et al., J Gen Virol. February 1991; 72 (Pt 2):359-67; Dietzschold et al., J. Virol. August 1990; 64(8):3804-9; Becker, Virus Genes. February 1990; 3(3):277-84; Prehaud et al., Virology. December 1989; 173(2):390-9 and Wang et al., Chin Med J (Engl). November 1989; 102(11):885-9, the disclosures of which are incorporated by reference in their entireties, may be used in the present invention.

A “polynucleotide” is a polymeric form of nucleotides of any length, which contain deoxyribonucleotides, ribonucleotides, and analogs in any combination. Polynucleotides may have three-dimensional structure, and may perform any function, known or unknown. The term “polynucleotide” includes double-, single-stranded, and triple-helical molecules. Unless otherwise specified or required, any embodiment of the invention described herein that is a polynucleotide encompasses both the double stranded form and each of two complementary forms known or predicted to make up the double stranded form of either the DNA, RNA or hybrid molecule.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracyl, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support.

An “isolated” polynucleotide or polypeptide is one that is substantially free of the materials with which it is associated in its native environment. By substantially free, is meant at least 50%, advantageously at least 70%, more advantageously at least 80%, and even more advantageously at least 90% free of these materials.

The invention further comprises a complementary strand to a rabies glycoprotein polynucleotide.

The complementary strand can be polymeric and of any length, and can contain deoxyribonucleotides, ribonucleotides, and analogs in any combination.

Hybridization reactions can be performed under conditions of different “stringency.” Conditions that increase stringency of a hybridization reaction are well known. See for examples, “Molecular Cloning: A Laboratory Manual”, second edition (Sambrook et al. 1989). Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where SSC is 0.15 M NaCl and 15 mM citrate buffer) and their equivalent using other buffer systems; formamide concentrations of 0%, 25%, 50%, and 75%; incubation times from 5 minutes to 24 hours; 1, 2 or more washing steps; wash incubation times of 1, 2, or minutes; and wash solutions of 6×SSC, 1×SSC, 0.1×SSC, or deionized water.

The invention further encompasses polynucleotides encoding functionally equivalent variants and derivatives of a rabies glycoprotein polypeptides and functionally equivalent fragments thereof which may enhance, decrease or not significantly affect properties of the polypeptides encoded thereby. These functionally equivalent variants, derivatives, and fragments display the ability to retain rabies glycoprotein activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan.

For the purposes of the present invention, sequence identity or homology is determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms can be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

In general, comparison of amino acid sequences is accomplished by aligning an amino acid sequence of a polypeptide of a known structure with the amino acid sequence of a the polypeptide of unknown structure. Amino acids in the sequences are then compared and groups of amino acids that are homologous are grouped together. This method detects conserved regions of the polypeptides and accounts for amino acid insertions and deletions. Homology between amino acid sequences can be determined by using commercially available algorithms (see also the description of homology above). In addition to those otherwise mentioned herein, mention is made too of the programs BLAST, gapped BLAST, BLASTN, BLASTP, and PSI-BLAST, provided by the National Center for Biotechnology Information. These programs are widely used in the art for this purpose and can align homologous regions of two amino acid sequences.

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Alternatively or additionally, the term “homology” or “identity”, for instance, with respect to a nucleotide or amino acid sequence, can indicate a quantitative measure of homology between two sequences. The percent sequence homology can be calculated as (Nref−Ndif)*100/Nref, wherein Ndif is the total number of non-identical residues in the two sequences when aligned and wherein Nref is the number of residues in one of the sequences. Hence, the DNA sequence AGTCAGTC will have a sequence identity of 75% with the sequence AATCAATC (Nref=8; Ndif=2).

Alternatively or additionally, “homology” or “identity” with respect to sequences can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur & Lipman, Proc Natl Acad Sci USA 1983; 80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being-considered equal to uracil (U) in RNA sequences.

And, without undue experimentation, the skilled artisan can consult with many other programs or references for determining percent homology.

The invention further encompasses a rabies glycoprotein contained in a vector molecule or an expression vector and operably linked to an enhancer and/or a promoter element if necessary. In an advantageous embodiment, the promoter is a cytomegalovirus (CMV) promoter. In another embodiment, the enhancers and/or promoters include various cell or tissue specific promoters, various viral promoters and enhancers and various rabies glycoprotein DNA sequences isogenically specific for each animal species.

A “vector” refers to a recombinant DNA or RNA plasmid or virus that comprises a heterologous polynucleotide to be delivered to a target cell, either in vitro or in vivo. The heterologous polynucleotide may comprise a sequence of interest for purposes of therapy, and may optionally be in the form of an expression cassette. As used herein, a vector need not be capable of replication in the ultimate target cell or subject. The term includes cloning vectors for translation of a polynucleotide encoding sequence. Also included are viral vectors.

The term “recombinant” means a polynucleotide of genomic cDNA, semisynthetic, or synthetic origin which either does not occur in nature or is linked to another polynucleotide in an arrangement not found in nature.

“Heterologous” means derived from a genetically distinct entity from the rest of the entity to which it is being compared. For example, a polynucleotide, may be placed by genetic engineering techniques into a plasmid or vector derived from a different source, and is a heterologous polynucleotide. A promoter removed from its native coding sequence and operatively linked to a coding sequence other than the native sequence is a heterologous promoter.

The polynucleotides of the invention may comprise additional sequences, such as additional encoding sequences within the same transcription unit, controlling elements such as promoters, ribosome binding sites, polyadenylation sites, additional transcription units under control of the same or a different promoter, sequences that permit cloning, expression, homologous recombination, and transformation of a host cell, and any such construct as may be desirable to provide embodiments of this invention.

Elements for the expression of rabies glycoprotein are advantageously present in an inventive vector. In minimum manner, this comprises, consists essentially of, or consists of an initiation codon (ATG), a stop codon and a promoter, and optionally also a polyadenylation sequence for certain vectors such as plasmid and certain viral vectors, e.g., viral vectors other than poxviruses. When the polynucleotide encodes a polyprotein fragment, e.g. rabies glycoprotein, advantageously, in the vector, an ATG is placed at 5′ of the reading frame and a stop codon is placed at 3′. Other elements for controlling expression may be present, such as enhancer sequences, stabilizing sequences and signal sequences permitting the secretion of the protein.

Methods for making and/or administering a vector or recombinants or plasmid for expression of gene products of genes of the invention either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6,312,683; U.S. patent application Ser. No. 920,197, filed Oct. 16, 1986; WO 90/01543; WO91/11525; WO 94/16716; WO 96/39491; WO 98/33510; EP 265785; EP 0 370 573; Andreansky et al., Proc. Natl. Acad. Sci. USA 1996; 93:11313-11318; Ballay et al., EMBO J. 1993; 4:3861-65; Felgner et al., J. Biol. Chem. 1994; 269:2550-2561; Frolov et al., Proc. Natl. Acad. Sci. USA 1996; 93:11371-11377; Graham, Tibtech 1990; 8:85-87; Grunhaus et al., Sem. Virol. 1992; 3:237-52; Ju et al., Diabetologia 1998; 41:736-739; Kitson et al., J. Virol. 1991; 65:3068-3075; McClements et al., Proc. Natl. Acad. Sci. USA 1996; 93:11414-11420; Moss, Proc. Natl. Acad. Sci. USA 1996; 93:11341-11348; Paoletti, Proc. Natl. Acad. Sci. USA 1996; 93:11349-11353; Pennock et al., Mol. Cell. Biol. 1984; 4:399-406; Richardson (Ed), Methods in Molecular Biology 1995; 39, “Baculovirus Expression Protocols,” Humana Press Inc.; Smith et al. (1983) Mol. Cell. Biol. 1983; 3:2156-2165; Robertson et al., Proc. Natl. Acad. Sci. USA 1996; 93:11334-11340; Robinson et al., Sem. Immunol. 1997; 9:271; and Roizman, Proc. Natl. Acad. Sci. USA 1996; 93:11307-11312. Thus, the vector in the invention can be any suitable recombinant virus or virus vector, such as a poxvirus (e.g., vaccinia virus, avipox virus, canarypox virus, fowlpox virus, raccoonpox virus, swinepox virus, etc.), adenovirus (e.g., canine adenovirus), herpesvirus, baculovirus, retrovirus, etc. (as in documents incorporated herein by reference); or the vector can be a plasmid. The herein cited and incorporated herein by reference documents, in addition to providing examples of vectors useful in the practice of the invention, can also provide sources for non-rabies glycoprotein proteins or fragments thereof, e.g., non-rabies glycoprotein proteins or fragments thereof, cytokines, etc. to be expressed by vector or vectors in, or included in, the compositions of the invention.

The present invention also relates to preparations comprising vectors, such as expression vectors, e.g., therapeutic compositions. The preparations can comprise, consist essentially of, or consist of one or more vectors, e.g., expression vectors, such as in vivo expression vectors, comprising, consisting essentially or consisting of (and advantageously expressing) one or more of a rabies glycoprotein polynucleotides and, advantageously, the vector contains and expresses a polynucleotide that includes, consists essentially of, or consists of a coding region encoding rabies glycoprotein, in a pharmaceutically or veterinarily acceptable carrier, excipient or vehicle. Thus, according to an embodiment of the invention, the other vector or vectors in the preparation comprises, consists essentially of or consists of a polynucleotide that encodes, and under appropriate circumstances the vector expresses one or more other proteins of rabies glycoprotein or a fragment thereof.

It is understood to one of skill in the art that conditions for culturing a host cell varies according to the particular gene and that routine experimentation is necessary at times to determine the optimal conditions for culturing rabies glycoprotein depending on the host cell. A “host cell” denotes a prokaryotic or eukaryotic cell that has been genetically altered, or is capable of being genetically altered by administration of an exogenous polynucleotide, such as a recombinant plasmid or vector. When referring to genetically altered cells, the term refers both to the originally altered cell and to the progeny thereof.

Polynucleotides comprising a desired sequence can be inserted into a suitable cloning or expression vector, and the vector in turn can be introduced into a suitable host cell for replication and amplification. Polynucleotides can be introduced into host cells by any means known in the art. The vectors containing the polynucleotides of interest can be introduced into the host cell by any of a number of appropriate means, including direct uptake, endocytosis, transfection, f-mating, electroporation, transfection employing calcium chloride, rubidium chloride, calcium phosphate, DEAE-dextran, or other substances; microprojectile bombardment; lipofection; and infection (where the vector is infectious, for instance, a retroviral vector). The choice of introducing vectors or polynucleotides will often depend on features of the host cell.

In a specific embodiment, the pharmaceutical composition is directly administered in vivo, and the encoded product is expressed by the vector in the host. The methods of in vivo delivery a vector encoding rabies glycoprotein (see, e.g., U.S. Pat. No. 6,423,693; patent publications EP 1052286, EP 1205551, U.S. patent publication 20040057941, WO 9905300 and Draghia-Akli et al., Mol Ther. December 2002; 6(6):830-6; the disclosures of which are incorporated by reference in their entireties) can be modified to deliver a rabies glycoprotein of the present invention to a dog. The in vivo delivery of a vector encoding rabies glycoprotein described herein can be accomplished by one of ordinary skill in the art given the teachings of the above-mentioned references.

Advantageously, the pharmaceutical and/or therapeutic compositions and/or formulations according to the invention comprise or consist essentially of or consist of an effective quantity to elicit a therapeutic response of one or more expression vectors and/or polypeptides as discussed herein; and, an effective quantity can be determined from this disclosure, including the documents incorporated herein, and the knowledge in the art, without undue experimentation.

One skilled in the art can determine the effective plasmid dose to be used for each immunization or vaccination protocol and species from this disclosure and the knowledge in the art.

In an embodiment, the pharmaceutical and/or therapeutic compositions and/or formulations according to the invention are administered orally. In a particularly advantageous embodiment, the oral compositions are administered as a bait drop. For example, the bait drop can comprise a polymer cube (1.25 inches by 0.75 inches) that is hollow. A sachet, or plastic packet, containing the rabies vaccine can be inserted into the hollow area of the bait and sealed with wax. The bait is attractive to raccoons and other wildlife, and strong enough to withstand distribution from airplanes flying at low altitude (e.g., about 500 feet). When a raccoon or other wildlife finds the bait and bites into it, the sachet ruptures, allowing the vaccine to enter the raccoon's mouth. Raccoons and other wildlife then become vaccinated against rabies by this oral route.

Also in connection with such a therapeutic composition, from the disclosure herein and the knowledge in the art, the skilled artisan can determine the number of administrations, the administration route, and the doses to be used for each injection protocol, without any undue experimentation.

In some embodiments, non-ionic hydrophilic surfactants having a high hydrophilic-lipophilic balance (HLB) value may be added to the disclosed formulations. This group comprises ethoxylated fatty acid monoesters of sorbitan (in particular 20 ethoxyl groups) (e.g. ethoxylated sorbitan monolaurate such as TWEEN 20®, ethoxylated sorbitan monopalmitate such as TWEEN 40®, ethoxylated sorbitan monostearate (such as TWEEN 60®, ethoxylated sorbitan monooleate such as TWEEN 80®, ethoxylated fatty alcohols (in particular 15-30 ethoxyl groups) (e.g. BRIJ 78®, BRIJ 98®, BRIJ 721®), ethoxylated fatty acids (in particular 15-30 ethoxyl groups) (e.g. MYRJ 49®, MYRJ 51®, MYRJ 52®, MYRJ 53®), non-ionic block-copolymers (e.g. polyoxyethylene/polyoxypropylene copolymer (POE-POP), such as LUTROL F127®, LUTROL F68®), and combinations thereof.

The disclosed formulation may include fatty acid esters of sorbitan (e.g. sorbitan monolaurate, like SPAN 20®, sorbitan monopalmitate, such as SPAN 40®, sorbitan monostearate, such as SPAN 60®, sorbitan tristearate, such as SPAN 65®, sorbitan monooleate, like SPAN 80®, sorbitan trioleate, like SPAN 85®, sorbitan monoisostearate, such as ARLACEL 987®, sorbitan isostearate, such as CRILL 6®), fatty acid esters of mannide (e.g. MONTANIDE 80®, mannide monooleate (such as ARLACEL A®), mannide dioleate, mannide trioleate, mannide tetraoleate), ethoxylated fatty acid esters of mannide (2, 3 or 4 ethoxyl groups) (e.g. MONTANIDE 888®, MONTANIDE 103®, ethoxylated mannide monooleate, ethoxylated mannide dioleate, ethoxylated mannide trioleate, ethoxylated mannide tetraoleate), and combinations thereof. The fatty acid may be oleate, palmitate, stearate, isostearate, laurate and combinations thereof.

In some embodiments, oils may be added to the disclosed formulations, including mineral oils, such as paraffin oil including isoparaffinic oil and/or naphtenic oil, squalane, pristane, polyisobutene oil, hydrogenated polyisobutene oil, polydecene oil, polyisoprene oil, polyisopropene oil and the like. Such oils may, for example, be those marketed under the name “MARCOL 52®” or “MARCOL 82®” (produced by Esso, France) or “DRAKEOL 6VR®” or “DRAKEOL 5®” “DRAKEOL 7®” (produced by Penreco, USA), “CLEAROL®” (produced by Sonneborn, USA), “Paraffin Oil Codex AAB2®” (produced by Aiglon, France), BLANDOL (produced by Sonneborn, USA), ONDINA 915 (produced by Shell, UK). The oil may also be a mixture of oils comprising at least 2 oils selected among the oils described herein, and in any proportion. The mixture of oils may also comprise at least one oil selected among the oils described above and at least one vegetable oil, and this vegetable oil represents from about 0.1% to about 33% of the oily phase, preferably from about 10% to about 25% v/v. These vegetable oils are unsaturated oils rich in oleic acid that are biodegradable and preferably liquid at the storage temperature (about +4° C.) or at least make it possible to give emulsions that are liquid at this temperature. For example the vegetable oil may be groundnut oil, nut oil, sunflower oil, safflower oil, soya oil, onager oil and the like.

### REFERENCES

Dean et al., “Potency Testing of Low Egg Passage Modified Live-Virus Rabies Vaccines”, II Am. J. Vet. Res., July 1961, pages 644-649.
Gaskell et al., “Duration of immunity (DOI)—The regulatory issues”, Vet. Microbiology 117 (2006) pages 80-85.
Bruckner et al., “Alternatives for Potency Testing of Veterinary Vaccines: Rabies Vaccine as a Model.”, IABS Symposium on Reduction of Animal Usage in the Development and Control of Biological Products, (1985), Develop. Biol. Standard, Vol. 64, pages 93-97.
WHO. Rabies vaccines: WHO position paper. Weekly epidemiological record. No. 32, 2010; 85. p. 209-320.
Hethcote, H W. An immunization model for a heterogeneous population. Theoretical Population Biology 1978; 14:338-349.
Slate, D., Algeo, T P, Nelson, K M, et al. Oral rabies vaccination in north america: opportunities, complexities, and challenges. PLOS 2009; 3(13)1-9, e529.
Fearneyhough M G, Wilson P J, Clark K A, et al. Results of an oral rabies vaccination program for coyotes. Journal of the American Veterinary Medical Association 1998; 212(4):498-502.
Moore, S M, Hanlon, C A. Rabies specific antibodies: Measuring surrogates of protection against fatal disease. PLOS Neg Trop Dis 2010. 4(3)e595.
Brown, L J, R. C. Rosatte, R C, Fehlner-Gardiner C, et al. Journal of Wildlife Diseases 2011; 47(1):182-194.

The invention will now be further described by way of the following non-limiting examples.

### EXAMPLES

### Example 1

### Production of RepliVax-WN-Rabies G in Helper Cells

The general process by which RepliVax-WN Rabies (909G) pseudo infectious virus (PIV) was produced is presented in FIG. 3 (for additional details, please see US 2011/0135686 A1, to Sanofi Pasteur). First, RepliVax-WNV Rabies (909G) cDNA was produced by replacing the WNV C gene with Rabies (909G; SEQ ID NO:l). The cDNA was then transcribed, and the resulting 909G RNA transfected into VEEV replicon-infected BHK-21 helper cells. The helper cells expressed C protein (supplied by VEEV replicon), WNV prM and E proteins (from 909G RNA), and the Rabies G protein (from 909G), which resulted in amplification of 909G PIV. The ΔprME-RabG (230G; SEQ ID NO:3) cDNA was then constructed by inserting rabies G in place prM & E. Finally, when C was removed from 230G, the resulting virus was ΔCprME-RabG (ΔC230G; SEQ ID NO:5). Using the procedure outlined above, 230G PIV were produced using the BHK-21 cells infected with the VEEV replicon. When normal cells (i.e. in vivo vaccinate cells; those not expressing VEEV replicon) were exposed to 909G PIVs, prM-E WN VLPs containing no genetic material were produced, as were rabies G proteins, resulting in both protection against rabies and enhanced protection against West Nile Virus. In partial contrast, when normal cells were exposed to 230G PIVs, no VLPs were formed (owing to absence of prM/E), but since rabies G proteins were produced, protection against rabies was observed. Importantly, no new RepliVax PIVs were formed when either PIV (909G or 230G) was administered to normal cells.

### Example 2

### Evaluation of the RepliVax-WN Rabies Constructs by Vaccination and Virulent Rabies Challenge in Mice

Although initial testing of candidate vaccines in non-target animals can be useful, for example, to confirm the potential of a vector to express in vivo a full-length peptide, the literature is replete with examples demonstrating protective immunity data cannot be extrapolated from a different species to the target species. For example, Dean et al. shows that it has long been recognized that vaccine safety and potency tests should be made in animals of the species for which the vaccine is to be used, i.e., if a rabies vaccine is to be a canine rabies vaccine, guinea pig or mouse tests are not adequate. Therefore, literature concerning mouse tests of a rabies immunological composition fail to anticipate or render obvious the claimed invention (e.g. a method for eliciting protective immunity against rabies, in a target animal, including a canine, comprising the steps of administering the immunological composition that was demonstrated effective in mouse). Further, Gaskell et al. shows that for a product to be labeled a vaccine, there must be challenge trials in the target species, and that a duration of protection of at least 1 year is required for a product to be labeled as a rabies vaccine. Therefore, literature concerning short term mouse tests of a rabies immunological composition fail to anticipate or render obvious the herein claimed invention. Finally, Bruckner et al. shows that mouse tests are not appropriate for determining the safety and efficacy of a canine rabies vaccine.

The foregoing notwithstanding, mice were vaccinated with PIVs produced using the constructs depicted in FIG. 2 according to Table 1:

TABLE 1
Mouse vaccination experimental design

| | | VACCI- | | | | |
|---|---|---|---|---|---|---|
| VACCINE | SUB | NATION | DOSE | VOLUME | ROUTE | MICE |
| A (909G) | A-1 | Day 0 | 5 × 10⁶ | 0.5 | IP | 20* |
| | A-2 | Day 0, 14 | | | | |
| B (230G) | B-1 | Day 0 | | | | |
| | B-2 | Day 0, 14 | | | | |
| C (ΔC230G) | C-1 | Day 0 | | | | |
| | C-2 | Day 0, 14 | | | | |
| D (230G + | D-1 | Day 0 | | | | |
| 909) | D-2 | Day 0, 14 | | | | |
| E (909) | E-1 | Day 0 | | | | |
| | E-2 | Day 0, 14 | | | | |
| F (Killed | F | Day 7, 14 | 1:10 | | | |
| Rabies | | | dilution | | | |
| Virus in | | | of serial | | | |
| Al(OH)₃) | | | | | | |

*10 mice terminally bled on Day 20 for Rabies Neutralizing Titers (RFFIT)
*10 mice Challenged on Day 21 with 16LD₅₀ of Rabies Virus

Based on RFFIT titers, the most immunogenic vaccine was vaccine B, however, according to mortality data (FIG. 4), vaccine D was slightly more effective (by one animal). Vaccine B was selected for additional study in the target animal: canine

### Example 3

### Vaccination of Dogs with ΔprME-RabG (230G) PIV

Based on the results in disclosed in Example 2, the construct with a double deletion of the prM and E genes, expressing rabies virus G protein (ΔprME-RabG 230G) was further evaluated by serology in dogs. This protocol was designed to evaluate safety and the serological response to the selected construct at two doses. Eighteen (18), four month-old purpose-bred beagles were randomly allocated into one of four treatment groups (n=6), using litter ID as the primary randomization factor. On Day 0 (26 Jul. 2010) all dogs were vaccinated according to Table 2.

TABLE 2
Dog vaccination experimental design

| | | | Route/ | |
|---|---|---|---|---|
| | | | Once/ | Dogs/ |
| Groups | Vaccine | Vaccine dose | Vol | Group |
| A | Replivax ΔprME-RabG 230G | 10⁷ FFU/ml | SC/1 | 6 |
| | (high dose) | | mL | |
| B | Replivax ΔprME-RabG 230G | 10⁶ FFU/ml | SC/1 | 6 |
| | (low dose) | | mL | |
| C | Replivax 230E | 10⁷ FFU/ml | SC/1 | 6 |
| | (Negative control - | | mL | |
| | empty WNV) | | | |
| D | Experimental Inactivated | Imrab3 | SC/1 | 6 |
| | Rabies Vaccine | commercial | mL | |
| | (Positive control) | release dose | | |

Animals were monitored, post-vaccination (within 1 hour) for acute systemic reactions and injection sites were examined and rectal temperatures recorded daily for 3 days thereafter. Blood was collected for rabies antibody titers as measured by Rapid Fluorescent Focus Inhibition Test (RFFIT) 2 weeks prior to vaccination for baseline and on Days 7, 14, 28, 49, 70, 99, 127, 163, and 253. Dogs pertaining to Group D (positive control group) were released on study Day 284. The remaining 18 dogs from Groups A, B and C were bled on Days 344 and 366. Approximately one year after vaccination, all dogs from Group A (High Dose) and three randomly selected dogs from Group C (Negative Control) were released and reassigned to the virulent rabies challenge study (disclosed in Example 4). The remaining dogs from Group B and C will remain under the current study number until termination of the study at a later date.

Results.

No injection site reactions (diffuse swelling, firm swelling, pain upon palpation or pruritus) were observed following vaccination. There were no clinically significant elevations in rectal temperature following vaccination. Serological response (RFFIT antibody titers) of the individual animals to the vaccination protocols were tabulated. All dogs were seronegative (≦0.2 IU/ml) prior to Day 0, and all dogs from Group C (negative control group) remained seronegative throughout the study. The individual and Group Average RFFIT antibody titer following vaccination for Groups A, B and D are shown in Table 3.

TABLE 3
Serum Rabies Antibody Titer (IU/ml) following vaccination.

| Gp | ID | D7 | D14 | D28 | D49 | D70 | D99 | D127 | D163 | D253 | D344 | D366 |
|---|---|---|---|---|---|---|---|---|---|---|---|---|
| A | CBCCTI | 2.9 | 12.9 | 2.9 | 1.1 | 0.9 | 0.9 | 1.1 | 0.9 | 0.9 | 1.5 | 0.7 |
| Replivax | CBCCTW | 14.1 | 28.2 | 4.0 | 1.1 | 0.9 | 0.9 | 0.5 | 0.9 | 1.2 | 0.9 | 0.7 |
| ΔprME- | CBDCAM | 2.9 | 2.9 | 2.9 | 1.0 | 1.0 | 1.1 | 1.1 | 0.7 | 0.8 | 0.9 | 0.7 |
| RabG | CBDCBG | 0.6 | 2.9 | 2.5 | 0.7 | 0.2 | 0.6 | 0.8 | 0.8 | 0.9 | 0.8 | 0.3 |
| 230G | CBDCCS | 0.5 | 0.5 | 0.5 | 0.6 | 0.2 | 0.4 | 0.8 | 0.8 | 0.8 | 0.5 | ≦0.2 |
| (high | CBDCDA | 2.4 | 2.5 | 3.2 | 1.5 | 0.9 | 0.8 | 1.1 | 0.9 | 0.8 | 0.8 | 0.7 |
| dose) | | | | | | | | | | | | |
| Average | | 3.9 | 8.32 | 2.67 | 1 | 0.68 | 0.78 | 0.9 | 0.83 | 0.9 | 0.9 | 0.55 |
| (+/−Std Dev) | | (5.11) | (10.68) | (1.17) | (0.32) | (0.38) | (0.25) | (0.24) | (0.08) | (0.15) | (0.33) | (0.23) |
| B | CBCCTV | 2.9 | 6.4 | 2.9 | 0.8 | 0.6 | 0.8 | 0.5 | 0.5 | 0.7 | 0.8 | 0.8 |
| Replivax | CBCCUD | ≦0.1 | 0.6 | 3.2 | 0.8 | 0.4 | 0.7 | 0.6 | 0.2 | 0.6 | 0.5 | 0.2 |
| ΔprME- | CBDCAF | ≦0.1 | ≦0.1 | 0.4 | 0.3 | 0.6 | 0.6 | 0.3 | ≦0.2 | 0.6 | 0.3 | ≦0.2 |
| RabG | CBDCAN | ≦0.1 | ≦0.1 | ≦0.1 | ≦0.2 | 0.4 | 0.3 | 0.4 | ≦0.2 | 0.2 | 0.3 | 0.2 |
| 230G | CBDCCV | 0.2 | 2.7 | 2.1 | 0.8 | 0.9 | 0.6 | 0.9 | 0.8 | 0.7 | 1.1 | 0.9 |
| (low | CBDCCZ | ≦0.1 | 2.0 | 0.6 | 0.4 | 0.7 | 0.7 | 0.3 | 0.2 | 0.6 | 0.2 | ≦0.2 |
| dose) | | | | | | | | | | | | |
| Average | | 0.58 | 1.98 | 1.55 | 0.55 | 0.6 | 0.62 | 0.5 | 0.35 | 0.57 | 0.53 | 0.42 |
| (+/−Std Dev) | | (1.14) | (2.41) | (1.35) | (0.28) | (0.19) | (0.17) | (0.23) | (0.25) | (0.19) | (0.35) | (0.34) |
| D | CBCCSF | 12.9 | 11.8 | 2.9 | 4.5 | 4.3 | 4.0 | 3.6 | 3.4 | 4.0 | — | — |
| Experimental | CBCCSY | 2.7 | 8.2 | 2.9 | 1.8 | 0.9 | 1.1 | 1.0 | 0.9 | 0.8 | — | — |
| Inactivated | CBCCTJ | 2.9 | 11.8 | 2.9 | 2.7 | 0.9 | 0.8 | 0.4 | 0.3 | 0.6 | — | — |
| Rabies | CBCCTS | 2.9 | 2.9 | 2.9 | ≦0.2 | 0.3 | 0.3 | ≦0.2 | ≦0.2 | 0.2 | — | — |
| Vaccine | CBCCUJ | 2.7 | 11.8 | 2.7 | 1.1 | 0.8 | 0.8 | 0.6 | 0.3 | 0.6 | — | — |
| (Positive control) | CBDCBD | 2.7 | 12.9 | 2.9 | 0.8 | 0.8 | 0.5 | 0.3 | ≦0.2 | 0.4 | — | — |

| | | | | | | | | | | | |
|---|---|---|---|---|---|---|---|---|---|---|---|
| Average | 4.47 | 9.9 | 2.87 | 1.85 | 1.33 | 1.25 | 1.02 | 0.88 | 1.1 | — | — |
| (+/−Std Dev) | (4.13) | (3.79) | (0.08) | (1.56) | (1.47) | (1.38) | (1.3) | (1.26) | (1.44) | — | — |

* Samples with a titer >0.2 IU/ml are considered positive.

Conclusions.

The Replivax construct with a double deletion of the prM and E genes, expressing rabies virus G protein (ΔprME-RabG 230G) at 10⁷ FFU/ml and at 10⁶ FFU/ml produced no evidence of local or systemic adverse events when administered via the subcutaneous route in dogs. Thus, the safety profile in this small group of dogs supports further investigation for its use in this species. All dogs in the high dose vaccine group seroconverted by Day 7 and maintained positive rabies titers through Day 344. Although two dogs in the high dose vaccine showed a decrease in titer (0.2 IU/ml) on Day 70, titers were maintained above 0.2 IU/ml afterwards and until Day 344. Dogs in the low dose vaccine group demonstrated a lower rate and a delayed onset of seroconversion in comparison to the high dose vaccine group. Five out of six dogs had titers above 0.2 IU/ml by Day 28 in the low dose vaccine group and one dog remained seronegative throughout the study, except between Days 70 and 127 in which titers ranged from 0.3 to 0.4. All dogs in Group D (positive control group) seroconverted by Day 7 and maintained titers>0.2 IU/ml except for two dogs that had a drop in titer≦0.2 by Day 49 and the other by Day 163.

The average group titers were equivalent between the high dose and positive control group despite the high titers from dog CBCCTW from the high dose group and CBCCSF from the positive control group on Days 7 and 14. All groups had a peak average titer on Day 14 followed by a decrease afterwards. Titers were sustained>0.2 IU/ml for the majority of the dogs in Groups A and D. In summary, the Replivax construct with a double deletion of the prM and E genes, expressing rabies virus G protein appears to be safe for use in dogs. The serological response to Replivax 230G at 10⁷ FFU/ml was superior to the serological response induced by the vaccine at 10⁶ FFU/ml. For the remaining study animals, blood for rabies antibody titers will be taken every 4 months until rabies challenge at a later date.

### Example 4

### Efficacy Evaluation of a WNV Expressing the Rabies G Protein Against a Rabies Challenge in Dogs

Six dogs from the high dose vaccine group and in three dogs from the negative control group approximately 1 year after vaccination (please see Example 3). Thus, a total of nine, 16 month old, purpose-bred beagles originally enrolled in the study disclosed in Example 3 were used for this study. These 9 dogs included Group A dogs (n=6) that were vaccinated with Replivax-WNV construct ΔprME-RabG 230G (expressing rabies G) at a dose of 10⁷ FFU/ml (high dose group) and 3/6 randomly selected dogs in Group C that were vaccinated with Replivax-WNV 230E (not expressing rabies G) at a dose of 10⁷ FFU/ml (negative control group). Dogs were vaccinated once with 1 ml of the assigned vaccine, via the subcutaneous route. Blood was collected for rabies antibody titers as measured by Rapid Fluorescent Focus Inhibition Test (RFFIT) at pre-determined days from all dogs following vaccination.

Approximately 370 days after vaccination the selected dogs were subject to virulent rabies challenge using 1 ml New York Strain 1 42.90 at a dilution of 1:100. Forty mice were used for challenge back titration. The challenge material was administered under anesthesia by the intramuscular route, into the left and the right frontalis muscles (0.5 ml into each muscle). Post-challenge, dogs were observed for 30 days for mortality or evidence of progressive neurological signs. Evidence of clinical signs of rabies was considered a criterion for humane euthanasia. Serum was obtained from all dogs immediately after euthanasia for RFFIT testing. A sample of the medulla oblongata was collected at necropsy and submitted for detection of rabies virus using direct immunofluorescence.

Results.

The detection threshold in the RFFIT test is 0.1 or 0.2 IU/ml depending on the testing session. Seroconversion was defined as a change from a negative antibody titer (under detection threshold) to a positive rabies antibody titer (above detection threshold). All dogs in the high dose group seroconverted by Day 7 after vaccination and all dogs in the negative control group remained seronegative throughout the study. The calculated 50% mouse lethal dose (MLD₅₀) of the challenge virus administered was 1.7 log₁₀ (50.1 MLD₅₀) in 0.03 ml. As 1 ml was administered to each dog, the dog dose was 3.2 log₁₀ MLD₅₀. The pre and post-challenge RFFIT titers, and post-challenge rabies fluorescent antibody results and morbidity/mortality data are shown in Table 4.

TABLE 4
Summary results

| | | Serology | | | Morbidity/ | |
|---|---|---|---|---|---|---|
| | | RFFIT (IU/ml)* | | | Mortality | |
| | | Pre- | Post- | Ab | Euthanasia | |
| | | challenge | challenge | results | due to signs | |
| Vaccine | | (D366 post- | (day of | Brain | of Rabies | Day |
| Group | ID | vac.) | euthanasia) | sample | infection | of death |
| Replivax- | CBCCTI | 0.7 | 3.2 | Neg. | No | 30 |
| WNV | CBCCTW | 0.7 | 10.0 | Neg. | No | 30 |
| Delta- | CBDCAM | 0.7 | 2.7 | Neg. | No | 30 |
| prME-RabG | CBDCBG | 0.3 | 1.1 | Neg. | No | 30 |
| 230G (test | CBDCCS | ≦0.2 | 1.8 | Neg. | No | 30 |
| vaccine) | CBDCDA | 0.7 | 3.4 | Neg. | No | 30 |
| Replivax | CBCCSG | ≦0.2 | ≦0.2 | Pos. | Yes | 12 |
| 230E | CBDCAX | ≦0.2 | ≦0.2 | Pos. | Yes | 15 |
| (-control) | CBDCAJ | ≦0.2 | 0.8 | Pos. | Yes | 17 |

*Samples with a titer >0.2 IU/ml are considered positive.

Conclusions.

The calculated dog challenge dose was approximately 3.4 log₁₀ MLD₅₀. All dogs in the negative control group developed clinical signs between Days 12 and 17, consistent with natural canine rabies infection, such as change in behavior, lethargy, excessive salivation, body tremors and ataxia. None of the dogs in the test vaccine group developed clinical signs of rabies infection during the 30 days post-challenge observation period. Further, all brain samples tested positive for rabies virus in the negative control group versus none in the test vaccine group. Collectively, these results validate the challenge model used in this study.

All dogs in the test vaccine group showed an anamnestic response as reflected by the rabies antibody titers post-challenge. One out of three dogs in the negative control group seroconverted at time of euthanasia (Day 17). It is possible that the first two dogs euthanized on Days 12 and 15 in the negative control group did not have enough time to mount an immune response against rabies virus. The results of this study demonstrate that dogs vaccinated via the subcutaneous route with a single-dose of Replivax-WNV construct ΔprME-RabG 230G at 10⁷ FFU/dog were protected against a virulent rabies challenge 1 year after vaccination. The remaining dogs from Example 3 enrolled in Group B (n=6; low dose Replivax-WNV AprME-RabG 230G) and Group C (n=3; Replivax 230E—Negative control empty WNV vector) will be kept and subjected to rabies challenge at a later date.

### Example 5

### Efficacy Evaluation of a WNV Expressing the Rabies G Protein Against a Rabies Challenge in Dogs—Low-Dose Group

Objective.

To determine the rabies antibody response to the 230G construct at two different doses up to 366 days post vaccination. An additional objective was to determine the protection afforded by the 230G construct at 10⁶ FFU/dog (low-dose), two years after a single vaccination against a rabies challenge in dogs. Most surprisingly and unexpectedly, a single subcutaneous dose of the 230G (at 10⁶ FFU/dog) provided greater than 80% protection against subsequent challenge by highly virulent rabies.

Materials/Methods.

Twenty-four (24), four month old, purpose-bred beagles were randomly assigned to four vaccine groups with six dogs in each group. Group A received ΔprME-RabG 230G (constructs described above) at a dose of 10⁷ FFU/ml (high dose group). Group B received ΔprME-RabG 230G at a dose of 10⁶ FFU/ml (low dose group). Group C (negative control) dogs were vaccinated with 10⁷ FFU/dog of construct AprME 230E (with the same prME deletion as 230G but not expressing the rabies G protein). Group D (positive control) dogs were vaccinated with an experimental inactivated rabies vaccine. Dogs were vaccinated subcutaneously once with 1 ml of the assigned vaccine. Blood was collected for rabies antibody titers as measured by Rapid Fluorescent Focus Inhibition Test (RFFIT) at pre-determined days from all dogs following vaccination.

Approximately 2 years after vaccination, nine dogs, six from Group B and the three remaining dogs from Group C were subject to virulent rabies challenge using 1 ml New York Strain 1 42.90 at a dilution of 1:100. The challenge material was administered under anesthesia by the intramuscular route, into the left and the right frontalis muscles (0.5 ml into each muscle). Forty mice were used for challenge back titration. Post-challenge, dogs were observed for 30 days for mortality or evidence of progressive neurological signs. Evidence of clinical signs of rabies was considered a criterion for humane euthanasia. Serum was obtained from all dogs immediately after euthanasia for RFFIT testing. Both brain hemispheres were collected at necropsy and the right hemisphere was submitted for detection of rabies virus using direct immunofluorescence.

Pre-Challenge Serology.

Seroconversion was defined as a change from a negative antibody titer (under detection threshold, i.e. ≦0.2 IU/ml) to a positive rabies antibody titer (>0.2 IU/ml). All dogs in the negative control group maintained rabies titer≦0.2 IU/ml prior to rabies challenge. Three out of six dogs in the low dose test vaccine group demonstrated negative rabies titers approximately 2 years after vaccination and the range of positive titers for the other three dogs was 0.3 to 0.7 IU/ml.

Challenge Back Titration in Mice.

The calculated 50% mouse lethal dose (MLD₅₀) of the challenge virus administered was 2.2 log₁₀ (158.5 MLD₅₀) in 0.03 ml. As 1 ml was administered to each dog, the dog dose was 3.96 log₁₀ MLD₅₀.

Post-Challenge Results.

The pre- and post-challenge RFFIT titers, and post-challenge rabies fluorescent antibody results and morbidity/mortality data are shown in Table 5 below.

TABLE 5
Summary results

| | | Serology | | | |
|---|---|---|---|---|---|
| | | RFFIT (IU/ml)* | | | Mor- |
| | | Pre- | | Rabies | bidity/ |
| | | challenge | Post- | fluorescent | Mortality* |
| | | (Day 752 | challenge | Ab | Day of |
| | | post- | (day of | results | death |
| Vaccine | | vacci- | eutha- | Brain | post- |
| Group | ID | nation) | nasia) | sample | challenge |
| Replivax- | CBCCTV | 0.5 | 2.0 | Negative | 30 |
| WNV Delta- | CBCCUD | ≦0.2 | 2.0 | Negative | 30 |
| prME-RabG | CBDCAF | ≦0.2 | 0.8 | Negative | 30 |
| 230G | CBDCAN | 0.3 | 1.5 | Negative | 30 |
| (Test Vac- | CBDCCV | 0.7 | 3.7 | Negative | 30 |
| cine Low | CBDCCZ | ≦0.2 | 2.9 | Positive | 13 |
| dose 10⁶ | | | | | |
| FFU/ml) | | | | | |
| Replivax | CBCCTX | ≦0.2 | ≦0.2 | Positive | 13 |
| 230E | CBDCCE | ≦0.2 | ≦0.2 | Positive | 17 |
| (Negative | CBDCCY | ≦0.2 | 0.4 | Positive | 12 |
| control - | | | | | |
| empty | | | | | |
| vector) | | | | | |

*All dogs euthanatized prior to Day 30 post-challenge demonstrated clinical signs of rabies infection.

Discussion/Conclusion.

All dogs in the negative control group developed clinical signs between Days 12 and 17 compatible with canine rabies infection, such as change in behavior, lethargy, salivation, facial twitching, difficulty to swallow, and limb paralysis. Dog CBDCCZ from the low dose test vaccine group developed paralysis of the lower jaw and salivation 13 days following rabies challenge. The other five dogs in the low dose vaccine group demonstrated no clinical abnormalities up to 30 days post-challenge. All dogs euthanatized up to 17 days post-challenge were positive for rabies fluorescent antibody testing and the remaining dogs euthanatized at the end of the study were negative for rabies fluorescent antibody testing in the brain tissue. Thus, correlating with the clinical signs observed during the post-challenge period.

The results of this study demonstrate that five out of six dogs vaccinated via the subcutaneous route with a single-dose of the ΔprME-RabG 230G construct at 10⁶ FFU/dog were protected against a virulent rabies challenge 2 years after vaccination.

### Example 6

### Pig Serology Results (ΔprME-RabG 230G)

Twenty Yorkshire crossbred, weaned, mixed sex pigs, approximately 14 weeks of age on Day 0. Pigs had never been vaccinated for rabies, were negative for rabies titers by Rapid Fluorescent Focus Inhibition Test (RFFIT), and no pigs exhibited any injection site abnormalities prior to vaccination on Day 0. Vaccinations were blinded and given intramuscularly on the right side of the neck, by personnel not making observations for the study, utilizing a 3 cc syringe and a 18ga X 1″ needle.

Two mL total volume ΔprME-RabG 230G construct was administered via IM (perpendicular to the muscles of the anterior half of the right side of the neck) as either 10⁷ FFU/ml (high dose) or 10⁶ FFU/ml (low dose). Study details are further summarized in the Tables, and FIG. 6 presents the overall results (RFFIT). As indicated by the graph, the peak response to the vaccine occurred between day 7 and 28 for both the high and low dose vaccines with a recorded peak titer on Day 14 for both. Moreover, the vaccine appeared to be very safe in the target animal.

TABLE 6
Vaccine Groups

| | | | Route/ | | |
|---|---|---|---|---|---|
| Groups | Vaccine | Vaccine dose | Once | Volume | No of pigs |
| A | Replivax | 10⁷ FFU/ml | IM | 2 ml | 10 |
| | (high dose) | | | | |
| B | Replivax | 10⁶ FFU/ml | IM | 2 ml | 10 |
| | (low dose) | | | | |

TABLE 7
Timeline.

| Day | Activity |
|---|---|
| Day −1 | Pre-screen blood collection (Baseline Serology) |
| Day 0-70 | Daily General Clinical Observations |
| Day 0 | Baseline Injection site observations |
| | and rectal temperature |
| | Vaccination |
| | Post-vaccination observation |
| Day 1 | Injection site observations and rectal temperature |
| Day 7 | Injection site observations and rectal temperature |
| | (injection site observations will be recorded weekly |
| | for any pig with reactions on Day 7) |
| | Blood collection serology |
| Day 14, 28, 49 | Blood collection serology |
| Day 70 | Blood collection serology, end of the study |

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method of eliciting in an animal a protective immune response against rabies comprising administering to said animal a therapeutically effective amount of a composition comprising at least one pharmaceutically or veterinarily acceptable carrier, excipient, or vehicle and a pseudoinfectious virus particle (PIV) produced using a recombinant virus containing a rabies G polynucleotide.

2. The method of claim 1 wherein the rabies G polynucleotide has the sequence as set forth in SEQ ID NO:11.

3. The method of claim 1 or 2 wherein the mammalian subject is a Canidae, a Felidae, or a Suidae.

4. The method of claim 1 or 2 wherein the mammalian subject is a dog, a bitch or a puppy.

5. The method of claim 1 or 2 wherein the mammalian subject is a cat or a kitten.

6. The method of claim 1 or 2 wherein the subject is a sow, a pig, or a piglet.

7. The method of claim 1 or 2, wherein the virus is a flavivirus.

8. The method of claim 7 wherein the flavivirus is deficient for expression of any one or combinations of a C, a prM, or an E flavivirus protein.

9. The method of claim 8 wherein the flavivirus is deficient for expression of the prM and E proteins.

10. The method of claim 8 wherein the flavivirus is deficient for expression of the C protein.

11. The method of claim 8 wherein the flavivirus is deficient for expression of the prM, E, and C proteins.

12. The method of any one of claim 1, 2, or 8-11 wherein the animal is protected against rabies for at least about 1 year after administration of the composition.

13. The method of claim 12 wherein the dose is administered IP or SC and in a dose range of about 10 μg to about 300 μg.

14. The method of claim 12 or 13 wherein the protection lasts for at least about 2 years.

15. The method of claim 14 wherein the protection lasts for at least about 3 years.
